(19) United States
(12) Patent Application Publication
(10) Pub. No.: US 2012/0201858 A1
(43) Pub. Date: Aug. 9, 2012

(54) Multiple-arm peptide compounds, methods of manufacture and use in therapy

(76) Inventors: Cheng Li, Fremont, CA (US); Mir Imran, Los Altos, CA (US)

(21) Appl. No.: 13/136,885

(22) Filed: Aug. 12, 2011

Related U.S. Application Data
(63) Continuation of application No. 10/664,697, filed on Sep. 16, 2003, now abandoned.

(51) Int. Cl. A61K 9/00; C07K 7/08; C08G 18/32; A61P 7/02; A61K 35/12; A61K 38/10; A61P 29/00; C08F 14/26; C08G 75/20

(52) U.S. Cl. 424/400; 525/54.1; 530/326; 424/93.7; 514/21.4

(57) ABSTRACT

The present invention concerns compositions of matter, pharmaceutical compositions and method to produce multiple arm peptides-substrate (MAP-S) as composites having molecules covalently bonded thereto which exhibit enhanced cell adhesion, attachment, proliferation and the like in vivo. The composites MAP-S described herein as compositions and implants improve and accelerate the healing process and tissue implant integration for vascular and soft tissue, joint, bone and combinations thereof.

### BACKGROUND OF THE INVENTION

### Field of the Invention

The present invention concerns peptide or protein molecules having multiple arms which contain specific sequences, domains or groups which are useful to produce improved cell adhesion, proliferation, migration and spreading, anti-inflammation, healing response, antithrombogenic effect and the like. The methods of synthesis of these molecules are described.

### REFERENCES

Some references of interest (in alphabetical order here) are discussed below and include the following:
1. R. S. Bhatnagar, et al., J. of Biomolecular Structure & Dynamics, 14: 547-60 (1997).
2. R. S. Bhatnagar, et al., Tissue Eng., 5: 53-65 (1999).
3. R. S. Bhatnagar, et al., “Biomaterials Regulating Cell Function and Tissue Development,” Materials Research Society, Symposium Proceedings, R. C. Thomsen et al., (ed), vol. 530, p. 43-54.
4. R. S. Bhatnagar, U.S. Pat. Nos. 5,354,736; 5,635,482, 5,958,428 and 6,268,348.
5. M. H. Dang, et al., U.S. Pat. No. 6,159,531.
6. M. H. Dang, et al., U.S. Ser. No. 10/017,193, filed Dec. 12, 2001, U.S. Patent Publication 20030113478, published Jun. 19, 2003.
7. D. H. Davis, et al., Biomaterials, 23, 4019-27 (2002).
8. S. K. Dickeson, et al., Cell. Mol. Life. Sci, 54, 556-66 (1998).
9. T. Gumpenberger, et al., Biomaterials, in Internet Publication (2003).
10. R. Haigh, et al, Biomaterials, 23, 3509-16 (2002).
11. M. J. Humphries, “Peptide sequences in matrix proteins recognized by adhesion receptors,” D. H. Rohrbach, R. Timpl (eds), San Diego: Academic Press, 289-308 (1993).
12. M. I. Janssen, et al., Biomaterials, 23, 4847-54 (2002).
13. L. Y. Koo, et al., Cell Science, 115: 1423 (2002).
14. T. Pakalns, et al., Biomaterials, 20, 2265-79 (1999).
15. J. J. Qian, et al., J. of Biomedical Materials Research, 31, 545-54 (1996).
16. L. V. Rudakov, et al., U.S. Ser. No. 09/935,417, filed Aug. 22, 2001, U.S. Patent Publication 20020062145, published May 23, 2002.
17. H. Shin, et al., Biomaterials, vol 24, pp 4353-64 (November 2003). Internet publication (and the 102 background references cited therein).
18. A. L. Sieminski, et al., Biomaterials, 21, 2233-41 (2000).
19. J. Y. Wong, Biomaterials, 23, 3865-70 (2002).

### Description of Related Art

Medical implants are often placed inside the body through invasive surgical procedures that can injure cells, tissues and organs. These injuries automatically trigger blood coagulation. The blood clot at the site of the injury stops the bleeding and provides temporary protection to the exposed wound site. The blood coagulation also serves as a short term platform for cells to attach, proliferate and migrate during the wound healing process. Furthermore, many chemical mediators are released during the wound healing process. Among the chemical mediators released are extra cellular matrixes (ECMs). The ECMs are proteins that stimulate cell adhesion, differentiation, proliferation and migration. These cell functions are very critical to the wound healing process and are mediated by cell adhesion molecules (CAMs), such as integrins, cadherins, selectins, etc., within the extracellular matrix (ECM). Cell ligand receptors within cell adhesion molecules regulate and interact with approaching cells. In many cases, these cell ligands are comprised of short peptide sequences, such as Arg-Gly-Asp (RGD) (SEQ ID NO: 2), found in many extracellular matrix proteins, including fibronectin, Arg-Glu-Asp-Val (REDV) (SEQ ID NO: 3), found in the type III connecting segment region of fibronectin, Tyr-Ile-Gly-Ser-Arg (YIGSR) (SEQ ID NO: 5), and found in laminin, GIAG (SEQ ID NO: 9) or GTPGPQGIAGQRGVV (also known as P-15) (SEQ ID NO: 1), found in collagen. It has been found that cell adhesion molecules (CAMs) exhibit molecular features that are specific for recognition by circulating cells in vivo. These molecular features enhance cell adhesion, migration, proliferation, differentiation and the like which accelerate the short term and long term healing of wounds. These short peptides and their coatings on the surface of medical implants are useful for wound healing of vascular tissue, soft tissue, joints, bone and the like.

Since most biomaterials interact with surrounding cells at the interface, a great deal of attention has been paid to the development of surface properties that promote desirable interactions between biomaterials and surrounding cells. The development of biomimetic materials greatly depends on an understanding of how cells organize and direct specific interactions at the interface, so that new biomimetic materials can recognize, support, promote and interact with living cells of the surrounding tissues.

Information about the identity of short peptide sequences derived from native extracellular matrix proteins and their ability to promote cell adhesion and proliferation through the targeting of specific cell membrane receptors has led to the development of biomaterials with surfaces that express these biologically active sequences. (M. J. Humphries, 1993). The biomimetic systems of the current art are usually described in terms of ligands useful for enhanced cell adhesion, proliferation and migration, the related ligand structures, cell-binding activity and selectivity, functional linking groups connecting the ligands to the surface of the substrate by covalent bonding and the covalent bonding processes.

### General Aspects

Biomemetic materials make it possible to regulate and control cellular interactions with implanted biomaterials at the molecular level. The receptor binding to the ligand that is externally presented from the biomemetic material determines the strength of the cell attachment to the implanted surface, the cell migration rate on or through the biomaterial and the extent of cytoskeletal organization. The biological responses depend on several factors, including but not limited to: receptor-ligand affinity, density of the ligand and spatial distribution and steric considerations of the ligand. Important design factors include, the spatial distribution, density and/or concentration of the active external peptides and the spacer (i.e. the linking structure between the substrate and the active ligands (e.g. peptides) which freely extend outward from the network (Shin, et al., 2003)).

Biomaterials play a very important role in most tissue engineering applications. Biomaterials can serve as a substrate to which cell populations migrate and attach, be implanted with a variety of specific cell or structure types, as a cell delivery vehicle and being utilized as a drug carrier to activate specific cellular functions (e.g. anti-inflammation, anti-thrombogenesis) in the local matrix (Shin, et al., 2003).

The useful biological activity of the specific active (typically short) peptide sequences (ligands) upon coupling to the substrate are retained. The modified peptide is flexible, experiences minimal steric hindrance and the terminal ligands are maximally configured to interact with the in vivo cellular environment. Bio-inert linear chains such as polyethylene glycol (of specific molecular weights) and some non-specific linear peptides are reported to have been placed between the solid phase surfaces and the active peptides (ligands) (Shin, et al., 2003).

### Ligands (R1 and Cell Binding, Proliferation and Migration

The role in cell binding of a β-bend is described within the triple helical region in collagen α1(I) chain by R. S. Bhatnagar, et al., 1997. The conformational preferences and biological activity of a synthetic 15-residue peptide, GTPGPQGIAGQRGVV (P-15) (SEQ ID NO: 1), are evaluated. A molecular mechanism is suggested for cell binding to collagen fibers based on a conformational transition in collagen molecules on the fiber surface.

The design of biomimetic habitats for tissue engineering with GTPGPQGIAGQRGVV (P-15) (SEQ ID NO: 1), a synthetic peptide analogue of collagen is described by R. S. Bhatnagar, et al., (1999). The construction of biomimetic environments is described with a synthetic peptide analogue of collagen. A synthetic peptide ligand P-15 (SEQ ID NO: 1) for collagen receptors is utilized to show 3-D colony formation, increased osteogenic differentiation and deposition of highly oriented and organized matrix by human dermal and gingival fibroblasts and by osteoblast like HOS cells. (R. S. Bhatnagar, et al., Vol. 530).

Synthetic compounds and compositions having enhanced cell binding are described. The focus of these U.S. patents is the use of peptide structures similar to GTPGPQGIAGQRGVV (P-15) (SEQ ID NO: 1). Organic biomaterials covalently bonded to a substrate include the amino acid residue-ILe-Ala -folded in a β-bend are useful for enhanced cell binding. P-15 (SEQ ID NO: 1) and smaller fragments thereof are described. (R. S. Bhatnagar US patents '736, '482, '428 and '348).

Ligand recognition by the I domain-containing integrins is described. Various integrins are used having active common amino acid domains, e.g. the metal ion-dependent adhesion site (MIDAS) motif. Human a₂ integrin and I domain bind the collagens laminin and echovirus 1. (S. K. Dickeson, et al., 1998).

Enhanced cell attachment is described for anorganic bone mineral in the presence of a synthetic peptide related to collagen. Human dermal fibroblasts are attached to anorganic bone mineral (ABM) particles. (J. J. Qian, et al., 1996). The attachment of cells is increased with increasing levels of GTPGPQGIAGQRGVV (P-15) polypeptide (SEQ ID NO: 1) on the surface of the ABM particles.

A coating with genetic engineered hydrophobin is described which promotes growth of fibroblasts on a hydrophobic solid when the RGD (SEQ ID NO: 2) sequence is present. PTFE was found to have improved growth of fibroblasts by coating the solid with genetically engineered SC3 hydrophobin. (M. I. Janssen, et al., 2002).

The cellular recognition of synthetic peptide amphiphiles in self-assembled monolayer films is described. Looped RGD (SEQ ID NO: 2) amphiphiles promote adhesion, spreading and cytoskeletal reorganization of melanoma and endothelial cells. (T. Pakalns, et al., 1999).

The immobilization of RGD (SEQ ID NO: 2) to <111> silicon surfaces for enhanced cell adhesion and proliferation is described. Surface chemistry and microstructure need to be controlled to regulate cell behavior on biomaterial surfaces. RGD (SEQ ID NO: 2) surfaces are examined for fibroblast adhesion and proliferation. (D. H. Davis, et al. 2002).

Identification and validation of a novel cell-recognition site (KNEED) (SEQ ID NO: 11) on the 8^{th} type III domain of fibronectin are described. Peptides containing the KNEED (SEQ ID NO: 11) sequence (of fibronectin) participate in cell attachment and spreading. (J. Y. Wong, et al., 2002).

The subject matter of some of these references overlaps with the following two topics:

### Covalent Linking Groups

Co-regulation of cell adhesion by nanoscale RGD (SEQ ID NO: 2) organization and mechanical stimulus is described where the backbone of polymethyl methylate has a comb-like structure. Improved cell adhesion proliferation and density are observed. (L V. Koo, et al., 2002).

Organic linkers and spacers are described in the surface treatment of articles using a low temperature plasma treatment. These treated articles are used as grafts or stents and have biocompatible coatings. (M. H. Dang, et al., U.S. 6,159,531 and M. H. Dang, et al. (2003)).

### Covalent Bonding to Substrate and Surface Modification

Coatings having biological activity and medical implants having a surface coating thereof and a method of manufacture using a low temperature plasma treatment is described. (M. H. Dang, et al., U.S. Pat. No. 6,159,531).

A multi-step method of forming a coating on a substrate such as a stent or a graft is described. The surface is treated with a plasma at or near atmospheric pressure. A bioactive/biocompatible coating and/or drug releasable coating is prepared. (Dang, et al., 2003).

Adhesion and proliferation of human endothelial cells on photochemically modified polytetrafluoroethylene (PTFE) is described. PTFE is surface modified by formation of C═O, C—OH, C—OOH and CNH₂ which improves cell adhesion and proliferation. (T. Gumpenberger, et al. Biomaterials in press—Internet publication, 2003).

Polymers are exposed to UV light in the presence of ammonia to study biomaterials-microvasculature interactions. Specific substrates include polytetrafluoroethylene and polyvinyl alcohol (PVA). This is a review which surveys work on reported biomaterial-microvasculature interactions with a focus on the use of biomaterials (containing, e.g. RGD (SEQ ID NO: 2), YIGSR (SEQ ID NO: 5), PDSGR (SEQ ID NO: 12), REDV (SEQ ID NO: 3), etc.) to regulate the structure and function of the microvasculature. (A. L. Sieminski, et al., 2000).

The synthesis and properties of amphiphilic networks 2: a differential scanning calorimetric study of poly(dodecyl methacrylate-stat-2,3 propandiol-1-methacrylate-stat-ethandiol dimethacrylate) networks and adhesion and spreading of dermal fibroblasts on these materials is described. Amphiphilic networks are utilized to produce hydrogels. Human skin fibroblasts are cultured on the hydrogels and observed to grow and spread. (R. Haigh, et al., 2002).

A composite expandable device for delivering into a vessel carrying blood is described. A coating is on the inner surface of the polymer sleeve which enhances cell growth on the sleeve. (L. V. Rudakov, et al., 2002).

Since cells interact with cell-binding peptides through cell adhesion domains that bind to localized regions within the molecules, the ability of cells to bind to the surface coated with the cell-binding peptide will be greatly affected by the orientation and conformation of the peptide on the surface relative to its cell adhesion domains. In addition, the number of cell adhesion domains within the peptide also influences cell behaviors on the surface, such as adhesion, spreading, growth, and migration (L. Y. Koo et al (2002).

All articles, references, U.S. patents, U.S. patent publications, U.S. patent applications, standards and the like are incorporated herein by reference in their entirety for background.

None of the references individually or jointly in combination with each other in any fashion teach or suggest the present invention.

From the above description, it is apparent that a need exists for an improved surface coating on implants to accelerate the in vivo healing process.

The present invention provides compositions of matter, pharmaceutical compositions, implants, methods of manufacture and methods of therapy to improve the cell adhesion, migration, proliferation and spreading involved with the healing process. Multiple arm peptides (MAP) of the present invention are a relatively large compared to most cell-binding peptides such as RGD (SEQ ID NO: 2), REDV (SEQ ID NO: 3) and YIGSR (SEQ ID NO: 5). Because of their large size, MAP peptides effectively provide the suitable molecular orientation and conformation for approaching cells. Their multiple cell adhesion domains on the multiple arms also greatly enhances the cell binding activity and selectivity. It is easier to synthesize mid-sized, branched peptides and attach them covalently onto different implant materials than large extra cellular matrix (ECM) proteins, such as fibronectin and collagen.

### SUMMARY OF THE INVENTION

Composites of multiple arm peptides (or multiple antigenic peptides) and a substrate (MAP-S) of the present invention are useful for enhanced attachment, adhesion, migration, growing, organizing and differentiation of in vivo cells. They include a covalent organic structure (compound) having ligands that promote cell adhesion, attachment, migration, proliferation, differentiation and the like. The substrate S optionally being a matrix or having a modified surface is inert, solid, hydrogel or liquid, flexible, rigid, porous and/or non-porous.

The present invention describes compositions of matter, pharmaceutical compositions and implants for the active structure MAP-S wherein MAP is a covalently bound organic structure which is covalently bound to a substrate S, wherein S selected from the group consisting of metal, alloy, ceramic, natural polymer, synthetic polymer, bioabsorbable polymer, and combinations and blends thereof, and the organic structure MAP is selected from:

(R)ₙ₊₁—(Z)ₙ—X

where n is selected from 1, 3, 7 and 15, which produces the following exemplary structures:

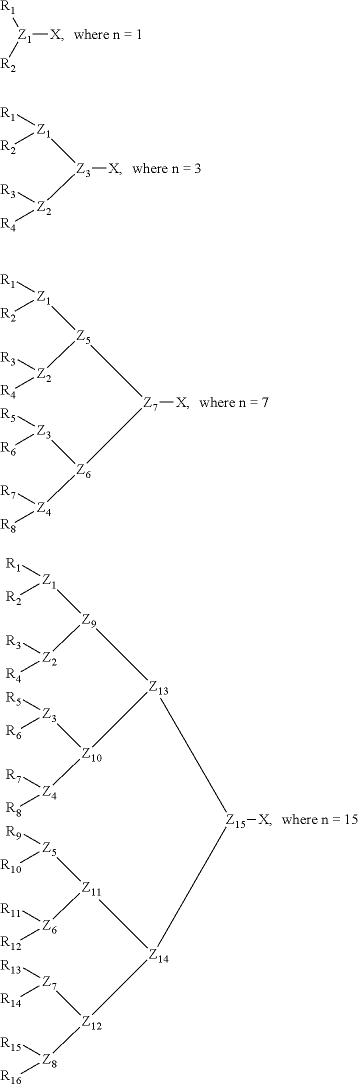

R and Z in each MAP structure are the same or a different moiety. Each R (i.e. R₁ to R₁₆ when present) may be any size or length and contains any type and number of cell-binding ligands and any type and number of other amino acids or anti-inflammatory or anti-thrombogenic structures. In addition, the MAP has at least one and optionally more than one active functional organic group to covalently link it to the surface of the substrate (S), where these active functional organic groups are a part of a group present: (i.e. a covalent part of X, Z or R).

X is an active or protected linking group selected from, but is not limited to, the group consisting of amine, one to five amino acids (X₁, X₂, X₃, X₄, X₅) which amino acids are the same or different, carboxylic acid, anhydride, hydroxyl, carbonyl, diol, disulfide (SH), hydroxyl succinimide (NHS) and siloxane;

Z (i.e., Z₁ to Z₁₅ when present) is independently selected from, but is not limited to, the group consisting of lysine, polylysine, ornithine or any known tri-functional organic or inorganic linkers; and

R (i.e., R₁ to R₁₆ when present) is independently selected from the group, but is not limited to,

(SEQ ID NO: 1)
GTPGPQGIAGQRGVV
or
P-15;
(SEQ ID NO: 2)
RGD
or
Arg-Gly-Asp;
(SEQ ID NO: 3)
REDV
or
Arg-Glu-Asp-Val;
(SEQ ID NO: 4)
C/H-V
or
WQPPRARI
or
Trp Gln Pro-Pro-Arg-Ala-Arg-Ile
(SEQ ID NO: 5)
YIGSR
or
Tyr-Ile-Gly-Ser-Arg;
(SEQ ID NO: 6)
SIKVAV
or
Ser-Ile-Lys-Val-Ala-Val;
(SEQ ID NO: 7)
F-9
or
RYVVLPRPVCFEKGMNYTVR
or
Arg-Tyr-Val-Leu-Pro-Arg-Pro-Val-Cys-Phe-
Glu-Lys-Gly-Met-An-Tyr-The-Val-Arg;
(SEQ ID NO: 8)
HEP-III
or
GEFYFDLRLKGDK
or
Gly-Glu-Phe-Tyr-Phe-Asp-Leu-Arg-Leu-Lys-Gly-Asp-
Lys;
(SEQ ID NO: 9)
GAG
or
Gly-Ile-Ala-Gly;
(SEQ ID NO: 10)
QGIAGQ
or
Gln-Gly-Ile-Ala-Gly-Gln;
(SEQ ID NO: 11)
KNEED
or
Lys-An-Glu-Glu-Asp;
(SEQ ID NO: 12)
PDSGR
or
Pro-Asp-Ser-Gly-Arg;

anti-inflammatory agents;
antithrombogenic agents; and
growth factor agents.

In one embodiment of the composition of matter, the substrate S is selected from a group consisting of hydroxylapatite, stainless steel, cobalt-chromium alloy, molybdenum alloy, titanium, titanium alloy, or a surface modified or unmodified polypropylene, polyethylene, polystyrene, polyether, polyamide/polyethylene copolymer, polychloroprene, polyester, polyvinyl chloride, polyolefin, polyphenolic, polyhydroxyacid, ABS epoxy, polytetrafluoro-ethylene, expanded polytetrafluoroethylene, polytetrafluoroethylene/polyethylene copolymer, fluorinated ethylene propylene, polyvinylidene, hexafluoropropylene, polyurethane, polysiloxane, polyisoprene, silicone, styrene butadiene, natural rubber, latex rubber, polyethyleneterephthalate, polycarbonate, polyamide, polyaramid, poly ether ketone, polyacetal, polyphenylene oxide, polysulfone, polyethersulfone, regenerated cellulose, polyamino acids, polyarylsulfone, polyphenylene sulfide, polybutylterephthalate (PBT) and combinations and blends thereof.

In one embodiment, in the composition of matter X is each independently selected from the group consisting of one to five amino acids (X₁, X₂, X₃, X₄ or X₅) and carboxylic acid.

In one embodiment, in the composition of matter Z₁ to Z₁₅ in each MAP structure when present is lysine.

In one embodiment, in the composition of matter Z₁ to Z₁₅ in each MAP structure when present is polylysine.

In one embodiment, in the composition of matter X₁ or X₂ are each independently selected from the group consisting of amino, amino acid, hydroxyl, and carboxylic acid.

Preferred MAP structures are those where n=1 (MAP2), n=3 (MAP4), n=7 (MAPS) and n=15 (MAP16). R (i.e. R₁ to R₁₆ when the specific R group is present) is selected from the group consisting of GTPGPQGIAGQRGVV (SEQ ID NO: 1), RGD (SEQ ID NO: 2), REDV (SEQ ID NO: 3), YIGSR (SEQ ID NO: 5), anti-inflammatory agents, anti-thrombogenic agents and combinations thereof.

Another important embodiment and advantage of the present invention is the use of amino acids, such as lysine, polylysine or ornithine as linkers to build the MAP structure. These structures are natural amino acids and are not expected to cause any detrimental in vivo effect (allergy, etc.) as may be likely with structures such as a polyethylene glycol, a conventional non-biological linking structure.

### BRIEF DESCRIPTION OF THE FIGURES

FIG. 1 is a graphic representation of human umbilical vein endothelial cells (HUVEC) growth of cells/cm² versus time in days.

FIG. 2 is a graphic representation of a second example of human umbilical vein endothelial cells (HUVEC) growth of cells/cm² versus time in days.

FIG. 3 is a graphic representation of human smooth muscle cells (HSMC) growth of cells/cm² versus time in days.

FIG. 4 is a schematic representation of the multiple arm peptide MAP2 where the R, X, Z and S groups are defined in the Summary of the Invention above.

FIG. 5 is a schematic representation of the multiple arm peptide MAP4 where the R, X, Z and S groups are defined in the Summary of the Invention above.

FIG. 6 is a schematic representation of the multiple arm peptide MAP8 where the R, X, Z and S groups are defined in the Summary of the Invention above.

FIG. 7 is a set of schematic MAP structures having specific amino acid lysine and alanine branching.

FIG. 8 is a schematic representation of direct synthesis and indirect synthesis of MAP structures.

FIG. 9 is a schematic representation of two different multiple arm peptides MAP8 connected to the surface of the substrate where the R, X, Z and S groups are defined in the Summary of the Invention.

### DETAILED DESCRIPTION OF THE INVENTION AND PREFERRED EMBODIMENTS

Novel branched multiple arm peptides (or multiple antigenic peptides) (MAPs) and those MAPs which are covalently bonded to a substrate (S) are described as compositions of matter and as components of implants in the present invention. The covalently bound MAPs have at least one terminus (and optionally more than one terminus) attached to a substrate (S) and multiple arms which terminate in the same or different organic groups which have a variety of biological functions in vivo. These functions include but are not limited to increased cell adhesion, attachment, migration, proliferation, differentiation and the like, anti-inflammation properties, anti-thrombogenic properties, growth factor properties and the like.

The enhanced biological activity of the MAP-S structure of the present invention is believed to be achieved by providing an array of freely rotating active peptide sequences which can alter their confirmation and orientation to expose active domains for cell attachment, adhesion and other biological effects. These compounds also have an increased density of active domains which are covalently bonded and do not migrate in vivo.

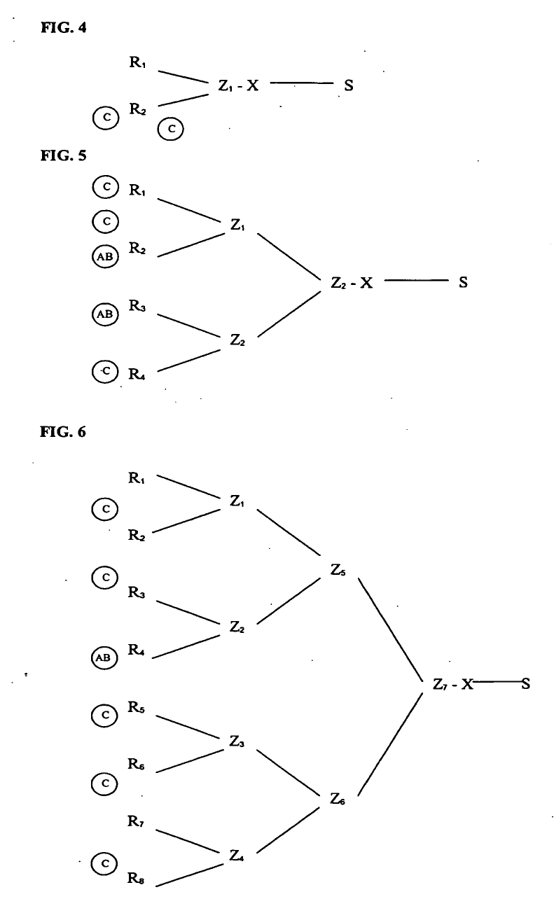

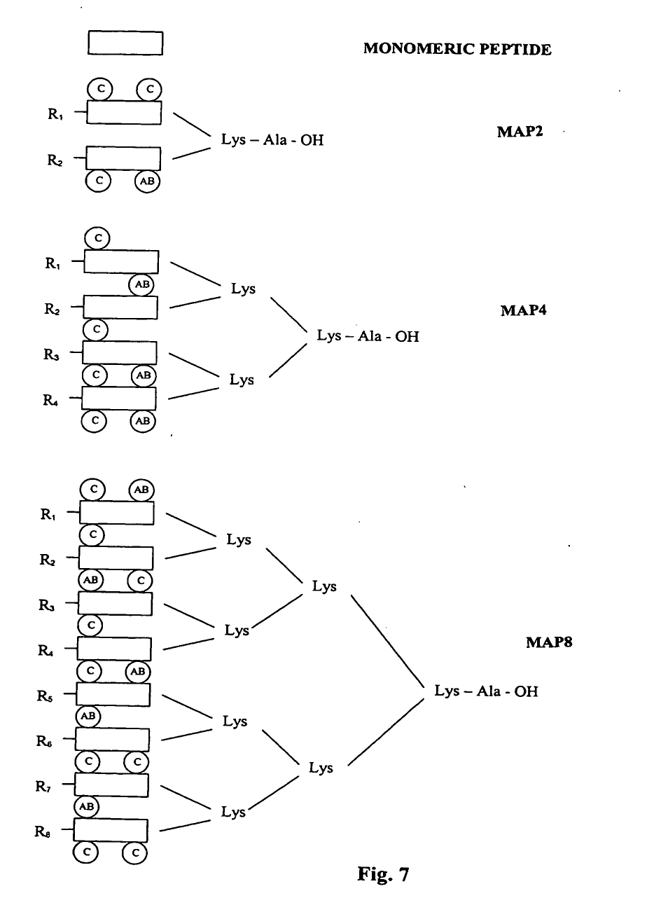

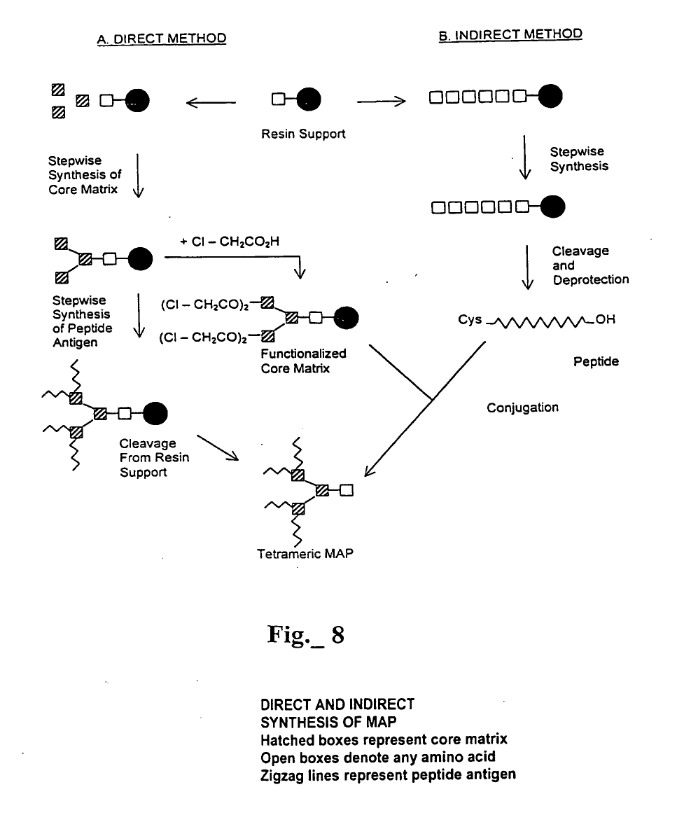

Referring now to FIGS. 4, 5, 6 and 7, FIG. 4 is a MAP2, FIG. 5 is a MAP4 and FIG. 6 is a MAPS. R, X, Z and S are described above in the Summary of the Invention. FIG. 7 shows all three MAPs as specific for lysine and alanine. As can be seen in each of these figures, the multiple arms extend out from the surface into open space are therefore exposed for the close approach and attachment of cells \textcircled{C}, and antibodies \textcircled{AB} and other useful biological factors in vivo.

The present invention also includes composites, implants and methods of use for promoting cell adhesion and other biological functions that comprise covalently attaching any of the above compositions of matter to a substrate (S, i.e. a matrix) and seeding living cells on the surface of the modified substrate. The substrates are listed above and in the Definitions which follow. Preferred substrates include biological or medical grade solids or biomaterials, i.e. those which are biologically compatible for in vivo applications and in vitro cell cultures. The invention is described in more detail below after the definitions of terms.

### DEFINITIONS AS USED HEREIN IN ALPHABETICAL ORDER INCLUDE

“Adhesive barrier” refers to those structures, which reduce the formation of connective tissue. See for example SEPRAFILM® adhesion barrier (a trademark of the Genzyme Corporation, Cambridge, Mass.). It is related to the polysaccharide hyaluronic acid found in connective tissue. (See also U.S. Pat. No. 4,851,521.)

“Anti-inflammatory agents” refers generally to smaller organic structures which are known to reduce a present inflammation in in vivo tissues. Structures include but are not limited to aspirin, ibuprofen, naproxen, aminoacetophen, COX-2 inhibitors (e.g. VIOX) and the like.

“Bioabsorbable polymer” refers to polymers of the art that can be used as the substrates S such as poly(glycolic acid) (PGA), poly(lactic acid) (PLA), PGA+PLA copolymers, poly(orthoesters), poly(p-dioxanone) (PDS), poly β-hydroxybutyrate (PHB), poly(PHB-hydroxyvaleric acid), pseudo-poly(amino acids), poly(iminocarbonates) and the like. See Y. H. An et al., Biomaterials, Vol. 21, 2675-2652 (2000). It is sometimes used interchangeably with the term biodegradable polymer.

“Biodegradable polymer” refers to polymers of the art that can be used as the substrate S, and includes but is not limited to poly(L-lactide) (LPLA), poly glycolide (PGA), poly(DL-lactide) (DLPLA), poly(dioxanone) (PDO), poly (DL-lactide-co-L-lactide) (LDLPLA), poly(DL-lactide-co-glycolide) (DLPLG), poly(glycolide-co-trimethylenecarbonate) (PGA-TMC), poly(L-lactide-co-glycolide) (LPLG), poly(epsilon-caprolactone) (PCL) and the like. (See J. C. Middleton et al., Biomaterials, vol. 21, 2335-2346 (2000).

“Cell-binding Sequence CR” or “cell binding domain CD” refer to amino acid sequences in a polypeptide or protein which enhance binding of living cells.

“Combinations” refers in relation to polymer structures, any combination including, but not limited to, copolymers, multiple polymers, blends, laminates, emulsions and the like.

“F-9” refers to RYVVLPRPVCFEKGMNYTVR or Arg-Tyr-Val-Leu-Pro-Arg-Pro-Val-Cys-Phe-Glu-Lys-Gly-An-Tyr-The-Val-Arg (SEQ ID NO: 7) (A. S. Charonis, et al. Cell Biol. 107: 1253 (1998).

“Growth factor” refers to those structures which are known to have growth enhancing properties for cells in vivo, generally for specific cell and/or tissue types. The term includes but is not limited to hepatocyte-growth factor (HGF), epidermal growth factor (EGF), erythropoietin (EPO), fibroblast growth factor (FGF), insulin-like growth factor (IGF), interleukins, nerve growth factor (NGF), platelet derived growth factor (PDGF), transforming growth factor (TGF), vascular endothelial growth factor (VEGF) and the like.

“HEP-Ilt or GEFYFDLRLKGDK” refers to Gly-Glu-Phe-Tyr-Phe-Asp-Leu-Arg-Leu-Lys-Gly-Asp-Lys (SEQ ID NO: 8) and a part of collagen (G. G. Koliakos, et al., J. Biol. Chem. 264: 2313-2332 (1989).

“Ligands” refers to the R groups of the structure herein (i.e. R₁ to R₁₆ when present in a MAP structure) which include structures which enhance cell adhesion, attachment, migration and proliferation, have anti-inflammatory properties, anti-thrombogenic properties, cell growth factor properties, adhesive barrier properties and the like. Different types of ligands R covalently bonded in the same MAP structure are contemplated.

“Optionally” refers to the invention when a component, bond, action, process step and the like may or may not be present. The invention is thus described whether or not that aspect is present or is not present.

“P-15” refers to GTPGPQGIAGQRCVV (SEQ ID NO: 1) and is found in collagen (R. S. Bhatnager, et al., Biomolec, Structure & Dynam, 14(5): 547-560 (1997) and R. S. Bhatnager, et al., Tissue Engineering 5(1): 53-65 (1999)).

“R₁ to R₁₆” refers in the structures in the Summary to various groups (ligands) having properties as peptides, anti-inflammatory agents, anti-thrombogenic agents and the like activity in in vitro and in vivo systems.

“REDV” refers to Arg-Glu-Asp-Val (SEQ ID NO: 3), which is found in the type III connecting segment region of fibronectin (J. A. Hubbell, et al., Ann. N.Y. Acad. Sd. 665: 253-258 (1993).

“RGD” refers to a tri-amino acid sequence of the structure -Arg-Gly-Asp- (SEQ ID NO: 2) which is found in many adhesive plasma and extracellular matrix proteins, including fibronectin (see for example Hynes, Cell, 11-25 (1992); Hubbell, et al., Bio/Technology, 9, 568-572, (1991); Massia, et al., J. Biomed. Mater. Res., 25, 223-242, 1999; and Lin, et al., J. Biomed. Mater., Res, 28, 329-342, 1994).

“Substrate (S)” refers to but is not limited to solid, hydrogel or liquid materials. Substrate refers to, for example, the following: polymer materials selected from hydrocarbons including polypropylene, polyethylene, polystyrene, polyether, polyamide/polyethylene copolymer, polychloroprene, polyester, polyvinyl chloride, polyolefin, polyphenolic, polyhydroxyacid, ABS epoxy, and corresponding copolymers and blends; fluorocarbons-including polytetrafluoroethylene, expanded polytetrafluorothylene, polytetrafluoroethylene/polyethylene copolymer, fluorinated ethylene propylene, polyvinylidene, hexafluoropropylene corresponding copolymers and blends; elastomers including polyurethane, polysiloxane, polyisoprene, silicone, styrene butadiene, natural rubber, latex rubber, and corresponding copolymers and blends; engineering thermoplastics including polyethyleneterephthalate, polycarbonate, polyamide, polyaramid, polyaryl ether ketone, polyacetal, polyphenylene oxide, polysulfone, polyethersulfone, regenerated cellulose, polyamino acids, polyarylsulfone, polyphenylene sulfide, polybutylphthalate (PBT), and the corresponding copolymers and blends thereof. Bioresorbable (or biodegradable) polymers such as poly(lactate) and poly(glycolide) are included (see above definitions). Hydrogel such as hydroxymethyl methacrylate (HEMA), hydroxymethyl acrylate, Di(hydroxymethyl) methacrylate, di(hydroxymethyl) acrylate, tri(hydroxyethyl) methacrylate, tri(hydroxymethyl) acrylate, and copolymers, blends and the like are included. Soluble polymers known in the art are also useful as substrate material. Mmetallic materials include stainless steel, cobalt-chromium-molybdenum alloy, pure titanium, and titanium alloys. In most applications the metallic and polymer materials have had their surfaces modified as is described herein to enhance the covalent bonding of the MAP structure.

“SIKVAV” refers to Ser-Ile-Lys-Val-Ala-Val (SEQ ID NO: 6) which is available from laminin (H. K. Kleimman, et al. Vitamins and Hormones 47: 161-186 (1993).

“YIGSR” refers to Tyr-Ile-Gly-Ser-Arg (SEQ ID NO: 5), which is found in laminin (S. P. Massia, et al., J. Biomed. Mater. Res., 25, 223-242, 1991).

“Z₁₋₁₅” refers in the structure in the Summary and claim 2 to various organic structures which are used to create the covalent multiple armed structure having the active terminal groups R₁-R₁₆. Preferred Z₁ to Z₁₅ groups (when present) include polyfunctional amino acids, such as lysine and polylysine.

The detailed description of the invention and preferred embodiments in R. S. Bhatnagar U.S. Pat. No. 5,354,736 is incorporated by reference here and it provides some useful description, preparations and background for the precursor peptides which are described in the present invention. Some precursor peptides as ligands are utilized in this invention in some MAP structures.

A suitable surface conformation is believed necessary for recognition by and the docking of living cells in vivo. The three-dimensional surface presented by the MAP region or parts of the MAP region are complementary to the reactive surface present on the cell-binding species (fibronectin). MAP compounds of the present invention mimic this surface ECM and any MAP compounds that can generate a similar surface are expected to have similar biological activity.

An embodiment of the present invention involves synthetic organic compositions of branched MAP structures that have enhanced biological activity functionally as compared to that of all or some portions of a single linear peptide chain. By “functionally comparable,” is meant that the shape, size and flexibility of a MAP compound is such that the biological activity of the MAP compound is enhanced when compared to the biological activity of the single linear peptide chain or a portion thereof. Of particular interest to the present invention utilizing branched MAP structures is the property of significantly enhanced cell binding as compared to that observed for small linear peptides. Useful ligands are selected on the basis of similar spacial and electronic properties as compared to the linear peptides or compounds. These individual ligands typically will be small molecules of amino acids of 100 or fewer or in the molecular weight range of up to about 10,000 daltons, and more typically up to 2,500 daltons. Inventive compounds are illustrated with synthetic MAP peptides; however, nonpeptide structures which mimic the necessary conformation for recognition and docking of cell-binding species are also contemplated as within the scope of this invention. For example, cyclic peptides or other compounds as R portions of the MAP structure in which the necessary conformation is stabilized by nonpeptides (e.g., thioesters) is one means of accomplishing the invention.

Of particular interest are the biological properties of the branched MAP compounds and composites which show increased cell binding in vitro of 50%, 100%, 250%, 500% or greater than is observed when compared to cell binding to a control surface. These in vitro results are a strong indicator that enhanced binding of the same magnitude or greater occurs and is useful in vivo. Each active terminal R group is preferably contemplated to be small covalently bonded molecules each of up to 50 amino acids or derivatives thereof. Examples of these small linear synthetic peptide groups as precursors for the ligands (R) of the MAP structures of the present invention include, but are not limited to, those found in Table 1 which includes sequence numbers.

TABLE 1
Linear Peptide Sequences as Precursors Provided
for Reference

| Description | SEQ ID NO: |
|---|---|
| GTPGPQGIAGQRGVV | 1 |
| RGD | 2 |
| REDV | 3 |
| C/H-V | 4 |
| WQPPRARI | |
| YIGSR | 5 |
| SIKVAV | 6 |
| RYVVLPRPVCFEKGMNYTVR | 7 |
| GEFYFDLRLKGDK | 8 |
| GIAG | 9 |
| QGIAGQ | 10 |
| KNEED | 11 |
| PDSGR | 12 |
| NH₂-GTPGPQGIAGQRGVV-lys-β-ala-COOH | 13 |

Examples of inventive MAP peptides are found in Table 2 with MAP identification numbers.

TABLE 2

| INVENTIVE PEPTIDES-MAP 2 STRUCTURES | MAP ID NO.: |
|---|---|
| (NH₂-Gly-Thr-Pro-Gly-Pro-Gln-Gly-Gln-Arg-Gly-Val-Val)₂-lys-β-ala-COOH | 13 |
| (CH₃CO-Gly-Thr-Pro-Gly-Pro-Gln-Gly-Gln-Arg-Gly-Val-Val)₂-lys-lys-(NH₂)-β-ala-CONH₂ | 14 |
| (CH₃CO-Gly-Thr-Pro-Gly-Pro-Gln-Gly-Gln-Arg-Gly-Val-Val)₂-lys-lys-(NH₂)-β-ala-COOH | 15 |
| (NH₂-Arg-Gly-Asp)₂-lys-β-ala-COOH | 16 |
| (CH₃CO-Arg-Gly-Asp)₂-lys-lys-(NH₂)-β-ala-CONH₂ | 17 |
| (CH₃CO-Arg-Gly-Asp)₂-lys-lys(NH₂)-β-ala-COOH | 18 |
| (NH₂-Arg-Glu-Asp-Val)₂-lys-βP-ala-COOH | 19 |
| (CH₃CO-Arg-Glu-Asp-Val)₂-lys-lys-(NH₂)-β-ala-CONH₂ | 20 |
| (CH₃CO-Arg-Glu-Asp-Val)₂-lys-lys-(NH₂)-β-ala-COOH | 21 |
| INVENTIVE PEPTIDES-MAP 4 STRUCTURES | MAP ID NO.: |
| (NH₂-Gly-Thr-Pro-Gly-Pro-Gln-Gly-Gln-Arg-Gly-Val-Val)₄-(lys)₂-lys-β-ala-COOH | 22 |
| (CH₃CO-Gly-Thr-Pro-Gly-Pro-Gln-Gly-Gln-Arg-Gly-Val-Val)₄-(lys)₂-lys-(NH₂)-β-ala-CONH₂ | 23 |
| (CH₃CO-Gly-Thr-Pro-Gly-Pro-Gln-Gly-Gln-Arg-Gly-Val-Val)₄-(lys)₂-lys-(NH₂)-β-ala-COOH | 24 |
| (NH₂-Arg-Gly-Asp)₄-(lys)₂-lys-β-ala-COOH | 25 |
| (CH₃CO-Arg-Gly-Asp)₄-(lys)₂-lys-lys-(NH₂)-β-ala-CONH₂ | 26 |
| (CH₃CO-Arg-Gly-Asp)₄-(lys)₂-lys-lys-(NH₂)-β-ala-COOH | 27 |
| (NH₂-Arg-Glu-Asp-Val)₄-(lys)₂-lys-β-ala-COOH | 28 |
| (CH₃CO-Arg-Glu-Asp-Val)₄-(lys)-lys-lys-(NH₂)-β-ala-CONH₂ | 29 |
| (CH₃CO-Arg-Glu-Asp-Val)₄-(lys)-lys-lys-(NH₂)-β-ala-COOH | 30 |
| INVENTIVE PEPTIDES-MAP 8 STRUCTURES | MAP ID NO.: |
| (NH₂-Gly-Thr-Pro-Gly-Pro-Gln-Gly-Gln-Arg-Gly-Val-Val)₈-(lys)₄-(lys)₂-lys-β-ala-COOH | 31 |
| (CH₃CO-Gly-Thr-Pro-Gly-Pro-Gln-Gly-Gln-Arg-Gly-Val-Val)₈-(lys)₄-(lys)₂-lys-lys-(NH₂)- | 32 |
| β-ala-CONH₂ | |
| (CH₃CO-Gly-Thr-Pro-Gly-Pro-Gln-Gly-Gln-Arg-Gly-Val-Val)₈-(lys)₄-(lys)₂-lys-lys-(NH₂)- | 33 |
| β-ala-COOH | |
| (NH₂-Arg-Gly-Asp)₈-(lys)₄-(lys)₂-lys-β-ala-COOH | 34 |
| (CH₃CO-Arg-Gly-Asp)₈-(lys)₄-(lys)₂-lys-lys-(NH₂)-β-ala-CONH₂ | 35 |
| (CH₃CO-Arg-Gly-Asp)₈-(lys)₄-(lys)₂-lys-lys-(NH₂)-β-ala-COOH | 36 |
| (NH₂-Arg-Glu-Asp-Val)₈-(lys)₄-(lys)₂-lys-β-ala-COOH | 37 |
| (CH₃CO-Arg-Glu-Asp-Val)₈-(lys)₄-(lys)₂-lys-lys-(NH₂)-β-ala-CONH₂ | 38 |
| (CH₃CO-Arg-Glu-Asp-Val)₈-(lys)₄-(lys)₂-lys-lys-(NH₂)-β-ala-COOH | 39 |
| INVENTIVE PEPTIDES-MAP 16 STRUCTURES | MAP ID NO.: |
| (NH₂-Gly-Thr-Pro-Gly-Pro-Gln-Gly-Gln-Arg-Gly-Val-Val)₁₆-(lys)₈-(lys)₄-(lys)₂-lys-β-ala-COOH | 40 |
| (CH₃CO-Gly-Thr-Pro-Gly-Pro-Gln-Gly-Gln-Arg-Gly-Val-Val)₁₆-(lys)₈-(lys)₄-(lys)₂-lys-lys-(NH₂)- | 41 |
| β-ala-CONH₂ | |
| (CH₃CO-Gly-Thr-Pro-Gly-Pro-Gln-Gly-Gln-Arg-Gly-Val-Val)₁₆-(lys)₈-(lys)₄-(lys)₂-lys-lys-(NH₂)- | 42 |
| β-ala-COOH | |
| (NH2-Arg-Gly-Asp)16-(lys)8-(lys)4-(lys)2-lys-β-ala-COOH | 43 |
| (CH₃CO-Arg-Gly-Asp)₁₆-(lys)₈-(lys)₄-(lys)₂-lys-lys-(NH₂)-β-ala-CONH₂ | 44 |
| (CH₃CO-Arg-Gly-Asp)₁₆-(lys)₈-(lys)₄-(lys)₂-lys-lys-(NH₂)-β-ala-COOH | 45 |
| (NH₂-Arg-Glu-Asp-Val)₁₆-(lys)₈-(lys)₄-(lys)₂-lys-β-ala-COOH | 46 |
| (CH₃CO-Arg-Glu-Asp-Val)₁₆-(lys)₈-(lys)₄-(lys)₂-lys-lys-(NH₂)-β-ala-CONH₂ | 47 |
| (CH₃CO-Arg-Glu-Asp-Val)₁₆-(lys)₈-(lys)₄-(lys)₂-lys-lys-(NH₂)-β-ala-COOH | 48 |

In theoretical studies those MAP peptides having the inventive sequences: MAP ID NO: 13, MAP ID NO: 14 and MAP ID NO: 15, etc. show a high potential for conformations useful for cell adhesion, etc. The branches once synthesized are covalently defined and with the details provided in this application have generally predictable in vitro and in vivo properties.

Synthetic MAP peptides of this invention may or may not have a core sequence that has -Ile-Ala- formed in a β-bend at physiological conditions as described by R. S. Bhatnagar U.S. Pat. No. 5,635,482. In most embodiments, this specific bond is not present.

The synthetic MAP compounds of this invention also have one or more of the following properties: they promote cell migration into porous lattices; they bind to collagen receptors; they induce metalloproteinases; they can down-regulate prolyl hydroxylase and collagen; they inhibit inflammation: and they inhibit thrombogenesis. The enumerated properties (including promotion of cell attachment) of synthetic peptides for the inventive family is utilized to convey these highly desirable properties to composites for a wide variety of uses. The down-regulation of prolyl hydroxylase is of particular interest because it represents a key step in collagen synthesis. This means that MAP compounds of the invention can be used as inhibitors of collagen synthesis to block formation of scar tissue and thus promote scarless healing.

MAP peptides of the invention are preferably also substantially free of blocking groups (which are often used during peptide synthesis), such as t-butyloxycarbonyl group (“BOC”).

Synthetic MAP peptides that have the desired biological activities may be produced at least of two general approaches.

Precursor polypeptides having fewer than about 100 amino acids, usually fewer than about 50 amino acids and more usually fewer than about 25, may be synthesized by the well-known Merrifield solid-phase chemical synthesis and modifications thereof method wherein amino acids are sequentially added to a growing chain, see B. Merrifield, J. Am. Chem. Soc., 85:2149-56 (1963) and B. Merrifield, “Solid Phase Peptide Synthesis” in Peptides: Synthesis, Structure and Applications, B. Gutte, (ed), Academic Press, New York, p. 93-169 (1995). Linear peptides may be chemically synthesized by manual means or by automation in commercially available synthesis equipment.

Since the use of relatively short, linear peptides as ligands R (i.e., R₁ to R₁₆ when present) is advantageous in performing the synthesis of MAPs described in the present invention, the peptides are preferably produced in quantity and will be free from contaminating substances, which are often found in peptides produced by recombinant techniques.

However, the linear synthetic peptides used as starting material for the MAP peptides of the present invention may also be synthesized by recombinant techniques involving the expression in cultured cells of recombinant DNA molecules encoding the gene for a desired portion for the α1(I) strand of collagen. The recombinant DNA procedures are well known in and available to one of skill in the art. DNA synthesis of specific peptide sequences is available from a variety of commercial services and is described in more detail in the R. S. Bhatnagar U.S. patents '736, '428, 482, and '348.

The present invention also includes composites and methods of use (as implants) for promoting mammalian cell adhesion comprising attaching any of the above-described compositions of matter to a substrate (that is, a matrix) and adding cells to the composite. Substrates include, but are not limited to, those listed in the Definitions section above. Some examples of composites MAP-S of this invention are shown below in Tables 3, 4, 5 and 6. It is understood that the functional group bond in X₁ to X₅ as described herein is the one that is covalently bonded (e.g. as a —COO— bond, a —CONH— bond, —NH— bond, etc.) to the surface of the substrate (S) as is described herein.

TABLE 3
Composites of the Invention Having Two (2) Arms

| | MAP | | | | | |
|---|---|---|---|---|---|---|
| # | ID | R | Z | X₁ | X₂ | S |
| 8A | 13 | R₁ = R₂ = NH₂-P-15 | Z₁ = lys | β-ala-COOH | — | e-PTFE |
| 8B | 14 | R₁ = R₂ = CH₃CO-P-15 | Z₁ = lys | Lys(NH₂) | β-ala-CONH₂ | e-PTFE |
| 8C | 15 | R₁ = R₂ = CH₃CO-P-15 | Z₁ = lys | Lys(NH₂) | β-ala-COOH | e-PTFE |
| 8D | 13 | R₁ = R₂ = NH₂-P-15 | Z₁ = lys | β-ala-COOH | — | polyethylene |
| 8E | 14 | R₁ = R₂ = CH₃CO-P-15 | Z₁ = lys | Lys(NH₂) | β-ala-CONH₂ | polyethylene |
| 8F | 15 | R₁ = R₂ = CH₃CO-P-15 | Z₁ = lys | Lys(NH₂) | β-ala-COOH | polyethylene |
| 8G | 13 | R₁ = R₂ = NH₂-P-15 | Z₁ = lys | β-ala-COOH | — | titanium alloy |
| 8H | 14 | R₁ = R₂ = CH₃CO-P-15 | Z₁ = lys | Lys(NH₂) | β-ala-CONH₂ | titanium alloy |
| 8I | 15 | R₁ = R₂ = CH₃CO-P-15 | Z₁ = lys | Lys(NH₂) | β-ala-COOH | titanium alloy |
| 8J | 13 | R₁ = R₂ = NH₂-P-15 | Z₁ = lys | β-ala-COOH | — | silicone |
| 8K | 14 | R₁ = R₂ = CH₃CO-P-15 | Z₁ = lys | Lys(NH₂) | β-ala-CONH₂ | silicone |
| 8L | 15 | R₁ = R₂ = CH₃CO-P-15 | Z₁ = lys | Lys(NH₂) | β-ala-COOH | silicone |
| 8M | 13 | R₁ = R₂ = NH₂-P-15 | Z₁ = lys | β-ala-COOH | — | polysulfone |
| 8N | 14 | R₁ = R₂ = CH₃CO-P-15 | Z₁ = lys | Lys(NH₂) | β-ala-CONH₂ | polysulfone |
| 8O | 15 | R₁ = R₂ = CH₃CO-P-15 | Z₁ = lys | Lys(NH₂) | β-ala-COOH | polysulfone |
| 8P | 13 | R₁ = R₂ = NH₂-P-15 | Z₁ = lys | β-ala-COOH | — | polyurethane |
| 8Q | 14 | R₁ = R₂ = CH₃CO-P-15 | Z₁ = lys | Lys(NH₂) | β-ala-CONH₂ | polyurethane |
| 8R | 15 | R₁ = R₂ = CH₃CO-P-15 | Z₁ = lys | Lys(NH₂) | β-ala-COOH | polyurethane |
| 8S | 16 | R₁ = R₂ = NH₂-RGD | Z₁ = lys | β-ala-COOH | — | ePTFE |
| 8T | 17 | R₁ = R₂ = CH₃CO-RGD | Z₁ = lys | Lys(NH₂) | β-ala-CONH₂ | ePTFE |
| 8U | 18 | R₁ = R₂ = CH₃CO-RGD | Z₁ = lys | Lys(NH₂) | β-ala-COOH | ePTFE |
| 8V | 16 | R₁ = R₂ = NH₂-RGD | Z₁ = lys | β-ala-COOH | — | polysulfone |
| 8W | 17 | R₁ = R₂ = CH₃CO-RGD | Z₁ = lys | Lys(NH₂) | β-ala-CONH₂ | polysulfone |
| 8X | 18 | R₁ = R₂ = CH₃CO-RGD | Z₁ = lys | Lys(NH₂) | β-ala-COOH | polysulfone |
| 8Y | 16 | R₁ = R₂ = NH₂-RGD | Z₁ = lys | β-ala-COOH | — | polyurethane |
| 8Z | 17 | R₁ = R₂ = CH₃CO-RGD | Z₁ = lys | Lys(NH₂) | β-ala-CONH₂ | polyurethane |
| 8AA | 18 | R₁ = R₂ = CH₃CO-RGD | Z₁ = lys | Lys(NH₂) | β-ala-COOH | polyurethane |
| 8AB | 16 | R₁ = R₂ = NH₂-RGD | Z₁ = lys | β-ala-COOH | — | HEMA |
| 8AC | 17 | R₁ = R₂ = CH₃CO-RGD | Z₁ = lys | Lys(NH₂) | β-ala-CONH₂ | HEMA |
| 8AD | 18 | R₁ = R₂ = CH₃CO-RGD | Z₁ = lys | Lys(NH₂) | β-ala-COOH | HEMA |
| 8AE | 19 | R₁ = R₂ = NH₂-RGDV | Z₁ = lys | β-ala-COOH | — | ePTFE |
| 8AF | 20 | R₁ = R₂ = CH₃CO-RGDV | Z₁ = lys | Lys(NH₂) | β-ala-CONH₂ | ePTFE |
| 8AG | 21 | R₁ = R₂ = CH₃CO-RGDV | Z₁ = lys | Lys(NH₂) | β-ala-COOH | ePTFE |
| 8AH | 19 | R₁ = R₂ = NH₂-RGDV | Z₁ = lys | β-ala-COOH | — | polysulfone |
| 8AI | 20 | R₁ = R₂ = CH₃CO-RGDV | Z₁ = lys | Lys(NH₂) | β-ala-CONH₂ | polysulfone |
| 8AJ | 21 | R₁ = R₂ = CH₃CO-RGDV | Z₁ = lys | Lys(NH₂) | β-ala-COOH | polysulfone |
| 8AK | 19 | R₁ = R₂ = NH₂-RGDV | Z₁ = lys | β-ala-COOH | — | polyurethane |
| 8AL | 20 | R₁ = R₂ = CH₃CO-RGDV | Z₁ = lys | Lys(NH₂) | β-ala-CONH₂ | polyurethane |
| 8AM | 21 | R₁ = R₂ = CH₃CO-RGDV | Z₁ = lys | Lys(NH₂) | β-ala-COOH | polyurethane |
| 8AN | 19 | R₁ = R₂ = NH₂-RGDV | Z₁ = lys | β-ala-COOH | — | poly(glycolide) |
| 8AO | 20 | R₁ = R₂ = CH₃CO-RGDV | Z₁ = lys | Lys(NH₂) | β-ala-CONH₂ | poly(glycolide) |
| 8AP | 21 | R₁ = R₂ = CH₃CO-RGDV | Z₁ = lys | Lys(NH₂) | β-ala-COOH | poly(glycolide) |

TABLE 4
Composites of the Invention Having Four (4) Arms

| | MAP | | | | | |
|---|---|---|---|---|---|---|
| # | ID | R | Z | X₁ | X₂ | S |
| 9A | 22 | R₁ = R₂ = R₃ = R₄ = NH₂-P-15 | Z₁ = Z₂ = Z₃ = lys | β-ala-COOH | — | e-PTFE |
| 9B | 23 | R₁ = R₂ = R₃ = R₄ = CH₃CO-P-15 | Z₁ = Z₂ = Z₃ = lys | Lys(NH₂) | β-ala-CONH₂ | e-PTFE |
| 9C | 24 | R₁ = R₂ = R₃ = R₄ = CH₃CO-P-15 | Z₁ = Z₂ = Z₃ = lys | Lys(NH₂) | β-ala-COOH | e-PTFE |
| 9D | 22 | R₁ = R₂ = R₃ = R₄ = NH₂-P-15 | Z₁ = Z₂ = Z₃ = lys | β-ala-COOH | — | polyurethane |
| 9E | 23 | R₁ = R₂ = R₃ = R₄ = CH₃CO-P-15 | Z₁ = Z₂ = Z₃ = lys | Lys(NH₂) | β-ala-CONH₂ | polyurethane |
| 9F | 24 | R₁ = R₂ = R₃ = R₄ = CH₃CO-P-15 | Z₁ = Z₂ = Z₃ = lys | Lys(NH₂) | β-ala-COOH | polyurethane |
| 9G | 22 | R₁ = R₂ = R₃ = R₄ = NH₂-P-15 | Z₁ = Z₂ = Z₃ = lys | β-ala-COOH | — | silicone |
| 9H | 23 | R₁ = R₂ = R₃ = R₄ = CH₃CO-P-15 | Z₁ = Z₂ = Z₃ = lys | Lys(NH₂) | β-ala-CONH₂ | silicone |
| 9I | 24 | R₁ = R₂ = R₃ = R₄ = CH₃CO-P-15 | Z₁ = Z₂ = Z₃ = lys | Lys(NH₂) | β-ala-COOH | silicone |
| 9J | 22 | R₁ = R₂ = R₃ = R₄ = NH₂-P-15 | Z₁ = Z₂ = Z₃ = lys | β-ala-COOH | — | polysulfone |
| 9K | 23 | R₁ = R₂ = R₃ = R₄ = CH₃CO-P-15 | Z₁ = Z₂ = Z₃ = lys | Lys(NH₂) | β-ala-CONH₂ | polysulfone |
| 9L | 24 | R₁ = R₂ = R₃ = R₄ = CH₃CO-P-15 | Z₁ = Z₂ = Z₃ = lys | Lys(NH₂) | β-ala-COOH | polysulfone |
| 9M | 25 | R₁ = R₂ = R₃ = R₄ = NH₂-RGD | Z₁ = Z₂ = Z₃ = lys | β-ala-COOH | — | e-PTFE |
| 9N | 26 | R₁ = R₂ = R₃ = R₄ = CH₃CO-RGD | Z₁ = Z₂ = Z₃ = lys | Lys(NH₂) | β-ala-CONH₂ | e-PTFE |
| 9O | 27 | R₁ = R₂ = R₃ = R₄ = CH₃CO-RGD | Z₁ = Z₂ = Z₃ = lys | Lys(NH₂) | β-ala-COOH | e-PTFE |
| 9P | 25 | R₁ = R₂ = R₃ = R₄ = NH₂-RGD | Z₁ = Z₂ = Z₃ = lys | β-ala-COOH | — | polyurethane |
| 9Q | 26 | R₁ = R₂ = R₃ = R₄ = CH₃CO-RGD | Z₁ = Z₂ = Z₃ = lys | Lys(NH₂) | β-ala-CONH₂ | polyurethane |
| 9R | 27 | R₁ = R₂ = R₃ = R₄ = CH₃CO-RGD | Z₁ = Z₂ = Z₃ = lys | Lys(NH₂) | β-ala-COOH | polyurethane |
| 9S | 25 | R₁ = R₂ = R₃ = R₄ = NH₂-RGD | Z₁ = Z₂ = Z₃ = lys | β-ala-COOH | — | silicone |
| 9T | 26 | R₁ = R₂ = R₃ = R₄ = CH₃CO-RGD | Z₁ = Z₂ = Z₃ = lys | Lys(NH₂) | β-ala-CONH₂ | silicone |
| 9U | 27 | R₁ = R₂ = R₃ = R₄ = CH₃CO-RGD | Z₁ = Z₂ = Z₃ = lys | Lys(NH₂) | β-ala-COOH | silicone |
| 9V | 25 | R₁ = R₂ = R₃ = R₄ = NH₂-RGD | Z₁ = Z₂ = Z₃ = lys | β-ala-COOH | — | polysulfone |
| 9W | 26 | R₁ = R₂ = R₃ = R₄ = CH₃CO-RGD | Z₁ = Z₂ = Z₃ = lys | Lys(NH₂) | β-ala-CONH₂ | polysulfone |
| 9X | 27 | R₁ = R₂ = R₃ = R₄ = CH₃CO-RGD | Z₁ = Z₂ = Z₃ = lys | Lys(NH₂) | β-ala-COOH | polysulfone |
| 9Y | 28 | R₁ = R₂ = R₃ = R₄ = NH₂-REDV | Z₁ = Z₂ = Z₃ = lys | β-ala-COOH | — | e-PTFE |
| 9Z | 29 | R₁ = R₂ = R₃ = R₄ = CH₃CO-REDV | Z₁ = Z₂ = Z₃ = lys | Lys(NH₂) | β-ala-CONH₂ | e-PTFE |
| 9AA | 30 | R₁ = R₂ = R₃ = R₄ = CH₃CO-REDV | Z₁ = Z₂ = Z₃ = lys | Lys(NH₂) | β-ala-COOH | e-PTFE |
| 9AB | 28 | R₁ = R₂ = R₃ = R₄ = NH₂-REDV | Z₁ = Z₂ = Z₃ = lys | β-ala-COOH | — | polyurethane |
| 9AC | 29 | R₁ = R₂ = R₃ = R₄ = CH₃CO-REDV | Z₁ = Z₂ = Z₃ = lys | Lys(NH₂) | β-ala-CONH₂ | polyurethane |
| 9AD | 30 | R₁ = R₂ = R₃ = R₄ = CH₃CO-REDV | Z₁ = Z₂ = Z₃ = lys | Lys(NH₂) | β-ala-COOH | polyurethane |
| 9AE | 28 | R₁ = R₂ = R₃ = R₄ = NH₂-REDV | Z₁ = Z₂ = Z₃ = lys | β-ala-COOH | — | silicone |
| 9AF | 29 | R₁ = R₂ = R₃ = R₄ = CH₃CO-REDV | Z₁ = Z₂ = Z₃ = lys | Lys(NH₂) | β-ala-CONH₂ | silicone |
| 9AG | 30 | R₁ = R₂ = R₃ = R₄ = CH₃CO-REDV | Z₁ = Z₂ = Z₃ = lys | Lys(NH₂) | β-ala-COOH | silicone |
| 9AH | 28 | R₁ = R₂ = R₃ = R₄ = NH₂-REDV | Z₁ = Z₂ = Z₃ = lys | β-ala-COOH | — | polysulfone |
| 9AI | 29 | R₁ = R₂ = R₃ = R₄ = CH₃CO-REDV | Z₁ = Z₂ = Z₃ = lys | Lys(NH₂) | β-ala-CONH₂ | polysulfone |
| 9AJ | 30 | R₁ = R₂ = R₃ = R₄ = CH₃CO-REDV | Z₁ = Z₂ = Z₃ = lys | Lys(NH₂) | β-ala-COOH | polysulfone |
| 9AK | 31 | R₁ = R₂ = R₃ = R₄ = NH₂-P-15 | Z₁ = Z₂ = Z₃ = lys | β-ala-COOH | — | HEMA |
| 9AL | 32 | R₁ = R₂ = R₃ = R₄ = CH₃CO-P-15 | Z₁ = Z₂ = Z₃ = lys | Lys(NH₂) | β-ala-CONH₂ | HEMA |
| 9AM | 33 | R₁ = R₂ = R₃ = R₄ = CH₃CO-P-15 | Z₁ = Z₂ = Z₃ = lys | Lys(NH₂) | β-ala-COOH | HEMA |
| 9AN | 31 | R₁ = R₂ = R₃ = R₄ = NH₂-P-15 | Z₁ = Z₂ = Z₃ = lys | β-ala-COOH | — | poly(glycolide) |
| 9AO | 32 | R₁ = R₂ = R₃ = R₄ = CH₃CO-P-15 | Z₁ = Z₂ = Z₃ = lys | Lys(NH₂) | β-ala-CONH₂ | poly(glycolide) |
| 9AP | 33 | R₁ = R₂ = R₃ = R₄ = CH₃CO-P-15 | Z₁ = Z₂ = Z₃ = lys | Lys(NH₂) | β-ala-COOH | poly(glycolide) |

TABLE 5
Composites of the Invention Having Eight (8) Arms

| | MAP | | | | | |
|---|---|---|---|---|---|---|
| # | ID | R | Z | X₁ | X₂ | S |
| 10A | 31 | R₁ to R₈ - all NH₂-P-15 | Z₁ to Z₇ all = lys- | β-ala-COOH | — | e-PTFE |
| 10B | 32 | R₁ to R₈ - all CH₃CO-P-15 | Z₁ to Z₇ all = lys- | lys-NH₂ | β-ala-CONH₂ | e-PTFE |
| 10C | 33 | R₁ to R₈- all CH₃CO-P-15 | Z₁ to Z₇ all = lys- | lys-NH₂ | β-ala-COOH | e-PTFE |
| 10D | 31 | R₁ to R₈ - all NH₂-P-15 | Z₁ to Z₇ all = lys- | β-ala-COOH | — | polyurethane |
| 10E | 32 | R₁ to R₈ - all CH₃CO-P-15 | Z₁ to Z₇ all = lys- | lys-NH₂ | β-ala-CONH₂ | polyurethane |
| 10F | 33 | R₁ to R₈- all CH₃CO-P-15 | Z₁ to Z₇ all = lys- | lys-NH₂ | β-ala-COOH | polyurethane |
| 10G | 31 | R₁ to R₈ - all NH₂-P-15 | Z₁ to Z₇ all = lys- | β-ala-COOH | — | silicone |
| 10H | 32 | R₁ to R₈ - all CH₃CO-P-15 | Z₁ to Z₇ all = lys- | lys-NH₂ | β-ala-CONH₂ | silicone |
| 10I | 33 | R₁ to R₈- all CH₃CO-P-15 | Z₁ to Z₇ all = lys- | lys-NH₂ | β-ala-COOH | silicone |
| 10J | 31 | R₁ to R₈ - all NH₂-P-15 | Z₁ to Z₇ all = lys- | β-ala-COOH | — | polysulfone |
| 10K | 32 | R₁ to R₈ - all CH₃CO-P-15 | Z₁ to Z₇ all = lys- | lys-NH₂ | β-ala-CONH₂ | polysulfone |
| 10L | 33 | R₁ to R₈- all CH₃CO-P-15 | Z₁ to Z₇ all = lys- | lys-NH₂ | β-ala-COOH | polysulfone |
| 10M | 31 | R₁ to R₈ - all NH₂-P-15 | Z₁ to Z₇ all = lys- | β-ala-COOH | — | Ti alloy |
| 10N | 32 | R₁ to R₈ - all CH₃CO-P-15 | Z₁ to Z₇ all = lys- | lys-NH₂ | β-ala-CONH₂ | Ti alloy |
| 10O | 33 | R₁ to R₈- all CH₃CO-P-15 | Z₁ to Z₇ all = lys- | lys-NH₂ | β-ala-COOH | Ti alloy |
| 10P | 34 | R₁ to R₈ - all NH₂-RGD | Z₁ to Z₇ all = lys- | β-ala-COOH | — | e-PTFE |
| 10Q | 35 | R₁ to R₈ - all CH₃CO-RGD | Z₁ to Z₇ all = lys- | lys-NH₂ | β-ala-CONH₂ | e-PTFE |
| 10R | 36 | R₁ to R₈- all CH₃CO-RGD | Z₁ to Z₇ all = lys- | lys-NH₂ | β-ala-COOH | e-PTFE |
| 10S | 34 | R₁ to R₈ - all NH₂-RGD | Z₁ to Z₇ all = lys- | β-ala-COOH | — | polyurethane |
| 10T | 35 | R₁ to R₈ - all CH₃CO-RGD | Z₁ to Z₇ all = lys- | lys-NH₂ | β-ala-CONH₂ | polyurethane |
| 10U | 36 | R₁ to R₈- all CH₃CO-RGD | Z₁ to Z₇ all = lys- | lys-NH₂ | β-ala-COOH | polyurethane |
| 10V | 34 | R₁ to R₈ - all NH₂RGD | Z₁ to Z₇ all = lys- | β-ala-COOH | — | silicone |
| 10W | 35 | R₁ to R₈ - all CH₃CO-RGD | Z₁ to Z₇ all = lys- | lys-NH₂ | β-ala-CONH₂ | silicone |
| 10X | 36 | R₁ to R₈- all CH₃CO-RGD | Z₁ to Z₇ all = lys- | lys-NH₂ | β-ala-COOH | silicone |
| 10Y | 34 | R₁ to R₈ - all NH₂-RGD | Z₁ to Z₇ all = lys- | β-ala-COOH | — | polysulfone |
| 10Z | 35 | R₁ to R₈ - all CH₃CO-RGD | Z₁ to Z₇ all = lys- | lys-NH₂ | β-ala-CONH₂ | polysulfone |
| 10AA | 36 | R₁ to R₈- all CH₃CO-RGD | Z₁ to Z₇ all = lys- | lys-NH₂ | β-ala-COOH | polysulfone |
| 10AB | 34 | R₁ to R₈ - all NH₂-RGD | Z₁ to Z₇ all = lys- | β-ala-COOH | — | Ti alloy |
| 10AC | 35 | R₁ to R₈ - all CH₃CO-RGD | Z₁ to Z₇ all = lys- | lys-NH₂ | β-ala-CONH₂ | Ti alloy |
| 10AD | 36 | R₁ to R₈- all CH₃CO-RGD | Z₁ to Z₇ all = lys- | lys-NH₂ | β-ala-COOH | Ti alloy |
| 10AE | 37 | R₁ to R₈ - all NH₂-REDV | Z₁ to Z₇ all = lys- | β-ala-COOH | — | e-PTFE |
| 10AF | 38 | R₁ to R₈ - all CH₃CO-REDV | Z₁ to Z₇ all = lys- | lys-NH₂ | β-ala-CONH₂ | e-PTFE |
| 10AG | 39 | R₁ to R₈- all CH₃CO-REDV | Z₁ to Z₇ all = lys- | lys-NH₂ | β-ala-COOH | e-PTFE |
| 10AH | 37 | R₁ to R₈ - all NH₂-REDV | Z₁ to Z₇ all = lys- | — | — | polyurethane |
| 10AI | 38 | R₁ to R₈ - all CH₃CO-REDV | Z₁ to Z₇ all = lys- | lys-NH₂ | β-ala-CONH₂ | polyurethane |
| 10AJ | 39 | R₁ to R₈- all CH₃CO-REDV | Z₁ to Z₇ all = lys- | lys-NH₂ | β-ala-COOH | polyurethane |
| 10AK | 37 | R₁ to R₈ - all NH₂-REDV | Z₁ to Z₇ all = lys- | β-ala-COOH | — | silicone |
| 10AL | 38 | R₁ to R₈ - all CH₃CO-REDV | Z₁ to Z₇ all = lys- | lys-NH₂ | β-ala-CONH₂ | silicone |
| 10AM | 39 | R₁ to R₈- all CH₃CO-REDV | Z₁ to Z₇ all = lys- | lys-NH₂ | β-ala-COOH | silicone |
| 10AN | 37 | R₁ to R₈ - all NH₂-REDV | Z₁ to Z₇ all = lys- | β-ala-COOH | — | polysulfone |
| 10AO | 38 | R₁ to R₈ - all CH₃CO-REDV | Z₁ to Z₇ all = lys- | lys-NH₂ | β-ala-CONH₂ | polysulfone |
| 10AP | 39 | R₁ to R₈- all CH₃CO-REDV | Z₁ to Z₇ all = lys- | lys-NH₂ | β-ala-COOH | polysulfone |
| 10AQ | 37 | R₁ to R₈ - all NH₂-REDV | Z₁ to Z₇ all = lys- | β-ala-COOH | — | Ti alloy |
| 10AR | 38 | R₁ to R₈ - all CH₃CO-REDV | Z₁ to Z₇ all = lys- | lys-NH₂ | β-ala-CONH₂ | Ti alloy |
| 10AS | 39 | R₁ to R₈- all CH₃CO-REDV | Z₁ to Z₇ all = lys- | lys-NH₂ | β-ala-COOH | Ti alloy |
| 10AT | 31 | R₁ to R₈ - all NH₂-P-15 | Z₁ to Z₇ all = lys- | β-ala-COOH | — | HEMA |
| 10AU | 32 | R₁ to R₈ - all CH₃CO-P-15 | Z₁ to Z₇ all = lys- | lys-NH₂ | β-ala-CONH₂ | HEMA |
| 10AV | 33 | R₁ to R₈- all CH₃CO-P-15 | Z₁ to Z₇ all = lys- | lys-NH₂ | β-ala-COOH | HEMA |
| 10AW | 31 | R₁ to R₈ - all NH₂-P-15 | Z₁ to Z₇ all = lys- | β-ala-COOH | — | poly(glycolide) |
| 10AX | 32 | R₁ to R₈ - all CH₃CO-P-15 | Z₁ to Z₇ all = lys- | lys-NH₂ | β-ala-CONH₂ | poly(glycolide) |
| 10AY | 33 | R₁ to R₈- all CH₃CO-P-15 | Z₁ to Z₇ all = lys- | lys-NH₂ | β-ala-COOH | poly(glycolide) |
| 10AZ | 31 | R₁ to R₈ - all NH₂-RGD | Z₁ to Z₇ all = lys- | β-ala-COOH | — | HEMA |
| 10BA | 32 | R₁ to R₈ - all CH₃CO-RGD | Z₁ to Z₇ all = lys- | lys-NH₂ | β-ala-CONH₂ | HEMA |
| 10BB | 33 | R₁ to R₈- all CH₃CO-RGD | Z₁ to Z₇ all = lys- | lys-NH₂ | β-ala-COOH | HEMA |
| 10BC | 31 | R₁ to R₈ - all NH₂-RGD | Z₁ to Z₇ all = lys- | β-ala-COOH | — | poly(glycolide) |
| 10BD | 32 | R₁ to R₈ - all CH₃CO-RGD | Z₁ to Z₇ all = lys- | lys-NH₂ | β-ala-CONH₂ | poly(glycolide) |
| 10BE | 33 | R₁ to R₈- all CH₃CO-RGD | Z₁ to Z₇ all = lys- | lys-NH₂ | β-ala-COOH | poly(glycolide) |

TABLE 6
Composites of the Invention Having Sixteen (16) Arms

| | MAP | | | | | |
|---|---|---|---|---|---|---|
| # | ID | R | Z | X₁ | X₂ | S |
| 11A | 40 | R₁ to R₁₆ - all NH₂-P-15 | Z₁ to Z₁₅ all = lys- | β-ala-COOH | — | e-PTFE |
| 11B | 41 | R₁ to R₁₆ - all CH₃CO-P-15 | Z₁ to Z₁₅ all = lys- | lys-NH₂ | β-ala-CONH₂ | e-PTFE |
| 11C | 42 | R₁ to R₁₆ - all CH₃CO-P-15 | Z₁ to Z₁₅ all = lys- | lys-NH₂ | β-ala-COOH | e-PTFE |
| 11D | 40 | R₁ to R₁₆ - all NH₂-P-15 | Z₁ to Z₁₅ all = lys- | β-ala-COOH | — | polyurethane |
| 11E | 41 | R₁ to R₁₆ - all CH₃CO-P-15 | Z₁ to Z₁₅ all = lys- | lys-NH₂ | β-ala-CONH₂ | polyurethane |
| 11F | 42 | R₁ to R₁₆ - all CH₃CO-P-15 | Z₁ to Z₁₅ all = lys- | lys-NH₂ | β-ala-COOH | polyurethane |
| 11G | 40 | R₁ to R₁₆ - all NH₂-P-15 | Z₁ to Z₁₅ all = lys- | β-ala-COOH | — | silicone |
| 11H | 41 | R₁ to R₁₆ - all CH₃CO-P-15 | Z₁ to Z₁₅ all = lys- | lys-NH₂ | β-ala-CONH₂ | silicone |
| 11I | 42 | R₁ to R₁₆ - all CH₃CO-P-15 | Z₁ to Z₁₅ all = lys- | lys-NH₂ | β-ala-COOH | silicone |
| 11J | 40 | R₁ to R₁₆ - all NH₂-P-15 | Z₁ to Z₁₅ all = lys- | β-ala-COOH | — | polysulfone |
| 11K | 41 | R₁ to R₁₆ - all CH₃CO-P-15 | Z₁ to Z₁₅ all = lys- | lys-NH₂ | β-ala-CONH₂ | polysulfone |
| 11L | 42 | R₁ to R₁₆ - all CH₃CO-P-15 | Z₁ to Z₁₅ all = lys- | lys-NH₂ | β-ala-COOH | polysulfone |
| 11M | 43 | R₁ to R₁₆ - all NH₂-RGD | Z₁ to Z₁₅ all = lys- | β-ala-COOH | — | e-PTFE |
| 11N | 44 | R₁ to R₁₆ - all CH₃CO-RGD | Z₁ to Z₁₅ all = lys- | lys-NH₂ | β-ala-CONH₂ | e-PTFE |
| 11O | 45 | R₁ to R₁₆ - all CH₃CO-RGD | Z₁ to Z₁₅ all = lys- | lys-NH₂ | β-ala-COOH | e-PTFE |
| 11P | 43 | R₁ to R₁₆ - all NH₂-RGD | Z₁ to Z₁₅ all = lys- | β-ala-COOH | — | polyurethane |
| 11Q | 44 | R₁ to R₁₆ - all CH₃CO-RGD | Z₁ to Z₁₅ all = lys- | lys-NH₂ | β-ala-CONH₂ | polyurethane |
| 11R | 45 | R₁ to R₁₆ - all CH₃CO-RGD | Z₁ to Z₁₅ all = lys- | lys-NH₂ | β-ala-COOH | polyurethane |
| 11S | 43 | R₁ to R₁₆ - all NH₂-RGD | Z₁ to Z₁₅ all = lys- | β-ala-COOH | — | silicone |
| 11T | 44 | R₁ to R₁₆ - all CH₃CO-RGD | Z₁ to Z₁₅ all = lys- | lys-NH₂ | β-ala-CONH₂ | silicone |
| 11U | 45 | R₁ to R₁₆ - all CH₃CO-RGD | Z₁ to Z₁₅ all = lys- | lys-NH₂ | β-ala-COOH | silicone |
| 11V | 43 | R₁ to R₁₆ - all NH₂-RGD | Z₁ to Z₁₅ all = lys- | β-ala-COOH | — | polysulfone |
| 11W | 44 | R₁ to R₁₆ - all CH₃CO-RGD | Z₁ to Z₁₅ all = lys- | lys-NH₂ | β-ala-CONH₂ | polysulfone |
| 11X | 45 | R₁ to R₁₆ - all CH₃CO-RGD | Z₁ to Z₁₅ all = lys- | lys-NH₂ | β-ala-COOH | polysulfone |
| 11Y | 46 | R₁ to R₁₆ - all NH₂-REDV | Z₁ to Z₁₅ all = lys- | β-ala-COOH | — | e-PTFE |
| 11Z | 47 | R₁ to R₁₆ - all CH₃CO-REDV | Z₁ to Z₁₅ all = lys- | lys-NH₂ | β-ala-CONH₂ | e-PTFE |
| 11AA | 48 | R₁ to R₁₆ - all CH₃CO-REDV | Z₁ to Z₁₅ all = lys- | lys-NH₂ | β-ala-COOH | e-PTFE |
| 11AB | 46 | R₁ to R₁₆ - all NH₂-REDV | Z₁ to Z₁₅ all = lys- | β-ala-COOH | — | polyurethane |
| 11AC | 47 | R₁ to R₁₆ - all CH₃CO-REDV | Z₁ to Z₁₅ all = lys- | lys-NH₂ | β-ala-CONH₂ | polyurethane |
| 11AD | 48 | R₁ to R₁₆ - all CH₃CO-REDV | Z₁ to Z₁₅ all = lys- | lys-NH₂ | β-ala-COOH | polyurethane |
| 11AE | 46 | R₁ to R₁₆ - all NH₂-REDV | Z₁ to Z₁₅ all = lys- | β-ala-COOH | — | silicone |
| 11AF | 47 | R₁ to R₁₆ - all CH₃CO-REDV | Z₁ to Z₁₅ all = lys- | lys-NH₂ | β-ala-CONH₂ | silicone |
| 11AG | 48 | R₁ to R₁₆ - all CH₃CO-REDV | Z₁ to Z₁₅ all = lys- | lys-NH₂ | β-ala-COOH | silicone |
| 11AH | 46 | R₁ to R₁₆ - all NH₂-REDV | Z₁ to Z₁₅ all = lys- | β-ala-COOH | — | polysulfone |
| 11AI | 47 | R₁ to R₁₆ - all CH₃CO-REDV | Z₁ to Z₁₅ all = lys- | lys-NH₂ | β-ala-CONH₂ | polysulfone |
| 11AJ | 48 | R₁ to R₁₆ - all CH₃CO-REDV | Z₁ to Z₁₅ all = lys- | lys-NH₂ | β-ala-COOH | polysulfone |
| 11AK | 46 | R₁ to R₁₆ - all NH₂-P-15 | Z₁ to Z₁₅ all = lys- | β-ala-COOH | — | HEMA |
| 11AL | 47 | R₁ to R₁₆ - all CH₃CO-P-15 | Z₁ to Z₁₅ all = lys- | lys-NH₂ | β-ala-CONH₂ | HEMA |
| 11AM | 48 | R₁ to R₁₆ - all CH₃CO-P-15 | Z₁ to Z₁₅ all = lys- | lys-NH₂ | β-ala-COOH | HEMA |
| 11AN | 46 | R₁ to R₁₆ - all NH₂-P-15 | Z₁ to Z₁₅ all = lys- | β-ala-COOH | — | poly(glycolide) |
| 11AO | 47 | R₁ to R₁₆ - all CH₃CO-P-15 | Z₁ to Z₁₅ all = lys- | lys-NH₂ | β-ala-CONH₂ | poly(glycolide) |
| 11AP | 48 | R₁ to R₁₆ - all CH₃CO-P-15 | Z₁ to Z₁₅ all = lys- | lys-NH₂ | β-ala-COOH | poly(glycolide) |
| 11AQ | 46 | R₁ to R₁₆ - all NH₂-RGD5 | Z₁ to Z₁₅ all = lys- | β-ala-COOH | — | HEMA |
| 11AR | 47 | R₁ to R₁₆ - all CH₃CO-RGD | Z₁ to Z₁₅ all = lys- | lys-NH₂ | β-ala-CONH₂ | HEMA |
| 11AS | 48 | R₁ to R₁₆ - all CH₃CO-RGD | Z₁ to Z₁₅ all = lys- | lys-NH₂ | β-ala-COOH | HEMA |
| 11AT | 46 | R₁ to R₁₆ - all NH₂-RGD | Z₁ to Z₁₅ all = lys- | β-ala-COOH | — | poly(glycolide) |
| 11AU | 47 | R₁ to R₁₆ - all CH₃CO-RGD | Z₁ to Z₁₅ all = lys- | lys-NH₂ | β-ala-CONH₂ | poly(glycolide) |
| 11AV | 48 | R₁ to R₁₆ - all CH₃CO-RGD | Z₁ to Z₁₅ all = lys- | lys-NH₂ | β-ala-COOH | poly(glycolide) |

The mode of attachment to the substrate is via covalent linkages. Covalent linkages include, but are not limited to, those involving ester, amide, anime or ether, see Carey et al., Advanced Organic Chemistry, Part B, Plenum Press, New York (1983). An exemplary method of covalent linkages involves peptides of the present invention with additions of amino acids at either the N-terminus or C-terminus to provide for binding or conjugation of the peptide to a solid phase or another protein.

Preferred types of cells to be adhered to the MAP structures include endothelial cells; however, most, if not all, cell types may be used.

Because endothelial cells play the central role of lining the unique vascular system in the living organism in the processes of the wound healing, it makes the in vitro use of endothelial cells as a model an important focus of biomaterial research. Since the original technique of cultivating human endothelial cells in vitro published in 1973 by E. A. Jaffe et al (J. Clin. Invest., 52, 2745ff (1973)), our knowledge of the endothelium has been altered from it being a passive barrier between the blood and the vessel wall to being a highly dynamic tissue with fundamental regulatory roles in numerous physiological processes (C. J. Kirkpatrick, Int. J. Microcirc, 17, 231ff (1997)).

The endothelium regulates four principal areas of biological function:

1. Hemostatic control is achieved by the endothelium to maintain a delicate balance between pro- and anti-thrombogenic signals.

2. The endothelium is involved in growth control by producing growth factors, such as platelet derived growth factor (PDGF) and basic fibroblast growth factor (bFGF) in response to cytokine stimulation.

3. The endothelium exerts a vital controlling function in vascular tone, principally by the synthesis of nitric oxide and prostacyclin as potent vasodilators.

4. The endothelium is a central regulator of the inflammatory response.

Because of these important regulatory functions of the endothelium, the promotion of endothelization on the surface of implants is vital for blood contacting devices. Therefore, the use of endothelial cells in the evaluation of new biomaterials and biosurfaces becomes very advantageous and imperative.

### Biological Testing and Results

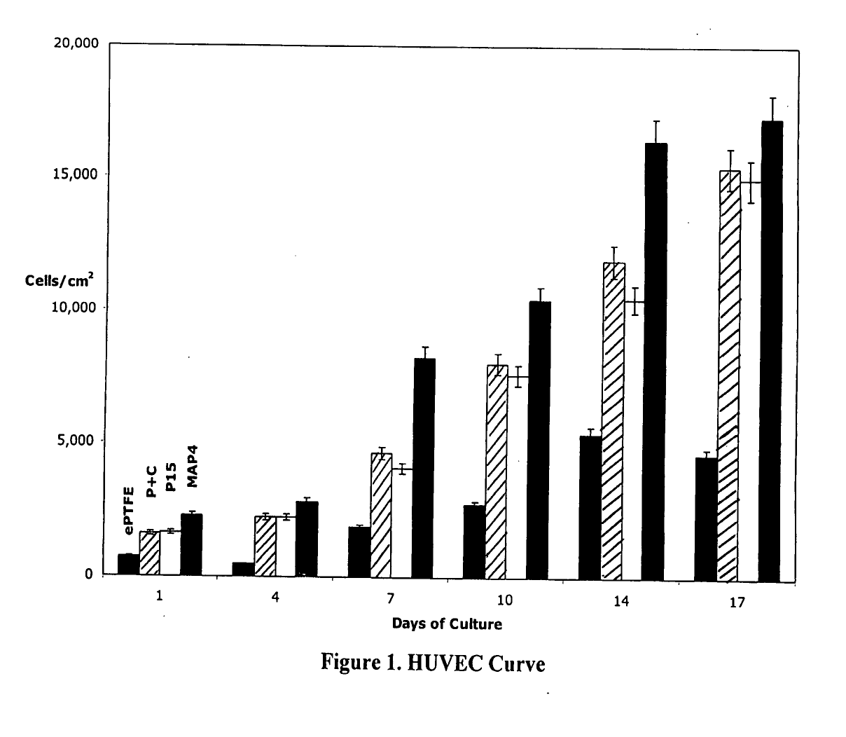

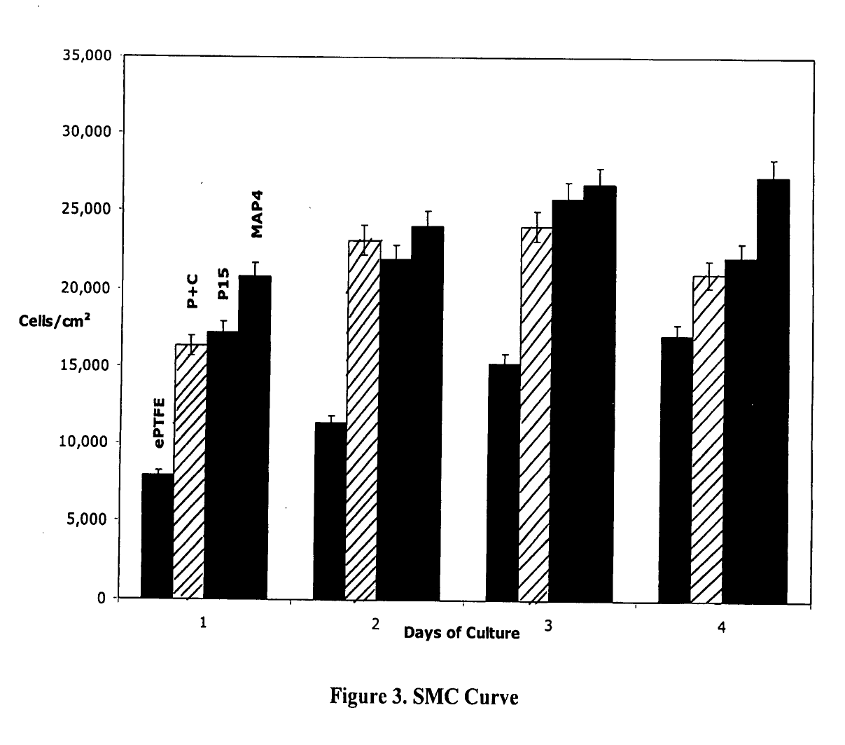

A more detailed description of the preparation and biological in vitro testing of FIGS. 1, 2 and 3 follows below.

In a specific embodiment, the present invention concerns peptide coated expanded polytetrafluoroethylene (ePTFE) stent graft materials in vitro using human umbilical vein endothelial cells (HUVEC). Four types of samples are studied: PTFE control (PTFE), chemical activated PTFE (P+C), GTPGPQGIAGQRGVV or (P-15) coated ePTFE (P-15), and MAP4 coated ePTFE (MAP4). Two identical experiments were carried out to obtain statistically reliable cell growth data. A similar plasma surface coating method described by M. H. Dang in U.S. Pat. No. 6,159,531 and also in U.S. patent publication 20030113478 (2003) was used to covalently bond peptides onto ePTFE films. The coated ePTFE samples were studied in vitro by seeding HUVEC onto their surfaces through a well established cell culture method. Results showed that MAP4 coated ePTFE had the largest increase in cell adhesion and cell proliferation over the ePTFE control. The cell adhesion of MAP4 coated ePTFE after 24 hours was more than 2.5 times better than that on ePTFE control. There were 400 percent more living cells on MAP4 coated ePTFE than that on ePTFE control after 4 days of incubation. MAP4 coated ePTFE was also more cell-promoting than P-15 coated ePTFE. It is believed that the superior cell-promoting properties exhibited by the MAP (e.g. MAP4) coating on ePTFE are due to its multiple cell adhesion domains within the MAP molecule and its large size to form suitable orientation and conformation for approaching cells. Similar cell growth data were found for both P-15 coated and chemical activated ePTFE samples.

Much of the discussion herein has focused in the use of small peptides as terminal ligands. Some specific features of the invention are described below with other uses and advantages then being apparent using the detailed information described herein to be apparent one of ordinary skill in the art.

### Multiple-Arm Peptide (MAP)—(Linking Groups)

The key feature of the MAP system is the many fold amplification of a peptide in a chemically defined manner. Unlike random polymerization which usually produces linear arrays of a wide range of polymers, the MAP polymer system produces branched covalently bonded polymers in a controlled manner having unambiguous structures. (In fact it was determined as part of these studies that a simple dimer of an active peptide, such as P-15-P-15, when tested showed little or no enhancement of cell adhesion, migration, differentiation or the like over the mono-peptide.) In the cascade type of MAP system, this structure is obtained by using a core matrix as a scaffolding consisting of several sequential ends of a tri-functional groups, such as an amino acid as a building unit. Lysine is the most commonly used because its two ends that amino acid groups are available for the branching. Other amino acids such as ornithine have also been used with success. When lysine is used, the core matric is unsymmetrical with a longer arm consisting of a side chain and a short arm consisting of the amino group. In the case of MAP 8 having three levels of branching, amino groups varying distance from 7 to 18 carbon atoms from the first branched carbon atom are observed. A symmetrical core is designed of lysine and alanine as a building unit. Further sequential propagation of lysine produces MAPs of tetravalent (MAP 4) or octavalent (MAP 8) or hexadecavalent (MAP 16), etc., reactive ends which are biologically active. MAP synthesis is now commercially available under contract with a number of companies. Usually, the MAP synthesis starts with a core of lysine-alanine or -lysine-lysine (NH₂)-ala- and builds the desired MAP structures.

A MAP structure was first described by J. P. Tam, Proc. Natl. Acad., SCl USA 85, 5409-13 (1988) and summarized by J. P. Tam, “Synthesis and Applications of Branched Peptides and Immunological Methods and Vaccines” in Peptides: Synthesis, Structures and Applications, B Sutte, (ed), Academic Press, San Diego, Calif., 455-500 (1995) and later reported by W. Huang et al., Mol. Immunol, 31, 1191-99 (1993), and MAP syntheses are also described by J. P. Tam in U.S. Pat. No. 5,580,563 and I. Toth et al. in U.S. Pat. No. 5,882,645, all of which are incorporated by reference in their entirety.

In one embodiment, the multiple arm peptides of the present invention are usually polypeptides (or protein) which have a high-density cluster of active terminal peptides which may account for over 90% of the total molecular weight of the MAP and which surround the smaller multi-branching lysine core. The backbone of this type of MAP is made up of amide bonds and typically these MAPs are remarkably stable in solution between pH 2 and 9. Thus, the MAPs can be prepared, stored and shipped as a lypholized powder.

With the lysine structure of this embodiment, a polarity preference is created when groups are attached to the core matrix for a C to N when it is synthesized in the conventional Merrifield type solid state synthesis. The conventional Boc-Benzyl-tert-butylcarbonyl chemistry or Fmoc-tert-butylfluorenyl-methoxycarbonyl chemistry is similar to that of a linear peptide with some modifications which within this application are with the skill in the art.

If a carboxyl group is being added, an indirect modular method is used. The two methods, direct and indirect, are shown in schematic form in FIG. 8. The indirect approach overcomes a lack of flexibility in the orientation of a peptide. It consists of the synthesis of a functionalized core matrix and unprotected peptides separately followed by chemoselective ligation of the two components. See for example J. P. Tam, 1995, p 460.

### Ligands Functioning as Anti-Inflammatory Agents

In another embodiment of this invention, the MAP motif is used to bind anti-inflammatory agents at the end of one or more of the branches present. Anti-inflammatory agents such as 2-acetoxybenzoic acid (aspirin), 2-(4-isobutylphenyl) propionic acid (ibuprofen), d-2-(6-methoxy-2-naphythyl) propionic acid (naproxen) and, COX-2 inhibitors (i.e. VIOX) are known anti-inflammatory agents used in a wide variety of therapeutic products. Each has a free unprotected carboxyl group which can be utilized by one of skill in the art in the Merrifield (1963) solid state synthesis described above.

In one embodiment, these anti-inflammatory agents are covalently coupled via the carboxylic acid to the free amine of the lysine to create an active amide bond. Thus, the terminal lysine in each of the short branches of MAP2, MAP4, MAP8 or MAP 16 is terminated with an anti-inflammatory group. Another embodiment is to increase the length of each amino acid branch of MAP with a number of standard amino acids (e.g., one to eight) then couple the anti-inflammatory agent to the final free amine group then in the terminal position by standard Merrifield solid state synthesis methods. Thus the R group in the MAP structure described herein has anti-inflammatory properties as the anti-inflammatory group is covalently coupled to 1 to 8 linking amino acids in each branch of the MAP.

### Ligands Functioning as Anti-thrombogenic Agents

In another embodiment of this invention, the MAP motif is used to bind anti-thrombogenic agents at the ends of one or more of the branches which are present. Suitable anti-thrombogenic agents include heparin (both high and low molecular weight), coumarin, hirudin (a polypeptide having a molecular weight of about 10,800) and its analogs and the like.

These agents are coupled through these agents' active functional groups (e.g. —NH₂) in the manner described above for the anti-inflammatory agents.

Alternatively, an amino acid chain of 1-8 amino acids is synthesized terminating in an amino group. Thus, the heparin amino group and the terminal amino group are then covalently coupled, using for example, an organic diacid, such as succinic acid and are ligands in the MAP structure.

### Ligands Functioning as Growth Factor Agents

In another embodiment of this invention, the MAP motif is used to covalently bind growth factor agents at the ends of one or more of the MAP branches which are present. Suitable growth factor agents include but are not limited to VEGF, PDGF and the other listed above in the Definitions.

These growth factors are coupled through these agents' active functional groups (e.g. —NH₂, —COOH, etc.) in the manner described above for the anti-inflammatory agents.

Alternatively, an amino acid chain of about 1-8 amino acids is synthesized terminating in an amino group. Thus, the growth factor amino group and the terminal amino group are then covalently coupled, using for example, an organic diacid, such as succinic acid and become ligands in the MAP structure.

### Ligands Functioning as Adhesive Barrier Agents

In another embodiment of this invention, the MAP motif is used to covalently bind adhesive barrier agents at the ends of one or more of the branches which are present. Suitable adhesive barrier agents include SEPRAFILM, DACRON or any of the materials known in the art to be used to combat the adhesion of unwanted cells or tissue.

These agents are coupled through these agents' active functional groups (e.g. —NH₂, —COOH, etc.) in the manner described above for the anti-inflammatory agents.

Alternatively, an amino acid chain of 1-8 amino acids is synthesized terminating in an amino group. Thus, surface amino groups of the adhesive barrier and the terminal amino group are then covalently coupled, using for example, an organic diacid, such as succinic acid and are ligands in the MAP structure.

### Formation of MAP-S

The synthetic MAP peptides are synthesized as is described herein. The MAP-structure (MAP-S) structure is formed by a number of processes. A preferred process is the plasma treatment described by M. H. Dang in U.S. Pat. No. 6,159,531 and also in US patent publication 20030113478 (2003). MAP peptides with active covalent linking groups are reacted with plasma treated and chemically activated substrate (S) (i.e., ePTFE or the other substrates listed in the Definitions above) as described in U.S. Pat. No. 6,159,531, which has organic functional groups on its surface. The resulting MAP-S article washed vigorously with water, ethanol or combinations thereof to remove free (non-covalently bonded) MAP peptides. The MAP article is then tested to confirm that the MAP-S covalently bonding to the surface coating has been obtained. Surface test methods include Amino Acid Analysis, MS, XPS, ATR-IR and other surface analytical techniques known in this art.

One MAP Structure having Different Ligands (R)

Another embodiment of the invention is to provide a MAP-S structure which have different ligands (R) on the same MAP structure. This is achieved by creating the first (MAP 2), second (MAP 4) and third generation (MAP 8) of the “tree” structure. Mixtures (in a ratio of about 50/50 or 33/66) of terminal ligands as a cell adhesion peptide R₁ and a second ligand R₂, for example, an anti-inflammatory agent are added and covalently coupled to create the mixed ligands R₁-, R₂—on the surface of the substrate. The amount of each of the different ligands which are covalently attached is determined by the reaction conditions, the ratios of the precursor R groups, type of R group, and attachment of both types of ligands R to the substrate S is observed.

### Two MAPs Having Different Ligands Attached to Substrate Surface

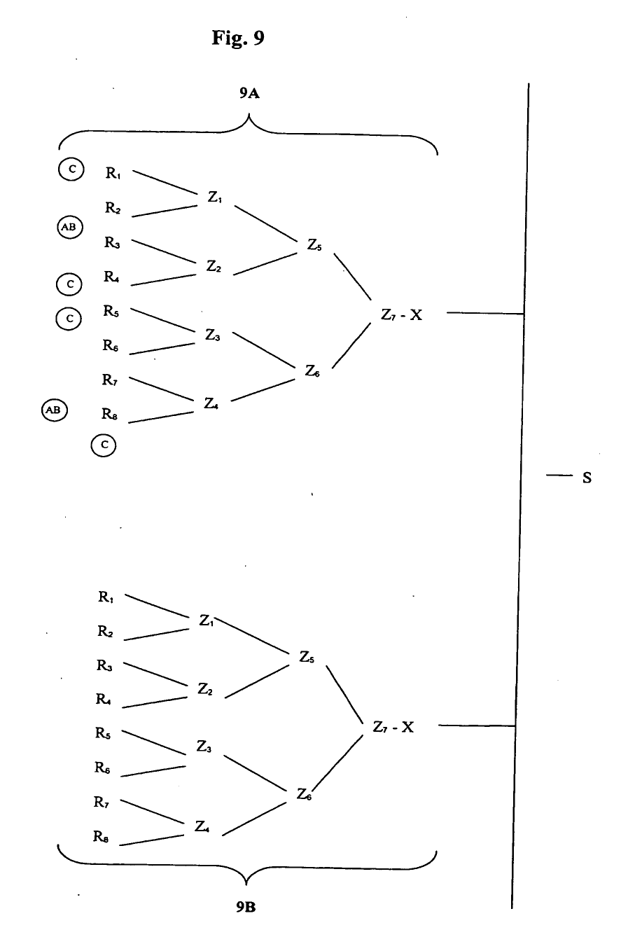

Another embodiment of this invention is to provide a MAP-S structure which has multiple biological properties. In this embodiment, two MAP compositions, one MAP (9A) having ligands selected from peptides useful for cell adhesion, and the other MAP (9B) having ligands useful as anti-inflammatory agents are covalently attached to a substrate (S) structure are prepared. (See FIG. 9) These two different MAP structures are then combined in a ratio of 50/50 to 33/66 and subjected to the standard covalent coupling described herein to the surface of a modified substrate S. The biological properties in vivo of the MAP-S article thus produced have both enhanced cell adhesion properties and enhanced anti-inflammation properties as compared to a single strand cell adhesion peptide or a single anti-inflammatory group. This approach is easily extended to produce and test the corresponding MAP8 and MAP16 structures.

Similarly three different ligand groups R₁, R₂ and R₃ cell migration groups are added to the surface of a treated substrate S by combining for example a 33/33/34 mixture of the R_{I}, R₂ and R₃ and immediately covalently coupling to the precursor MAP4, MAP8 and MAP 16 structure as is described above.

### MAP Crosslinking to Substrate S

Because of the many structures possible in the MAP2, MAP4, MAP8 and MAP16, it is also possible to synthesize structures which have multiple active covalent bonding sites 2, 3, 4, etc. for attaching to the substrate S. Thus, in the covalent MAP structures shown in the Summary above R and Z in addition to X may have an active (unprotected) functional group (such as an amine, amide, or carboxylic acid) which will, under the proper circumstances, also covalently bond to substrate (S). This creates an organic cross linked structure.

### MAP-S as a Pharmaceutical Composition

In another embodiment, this invention also includes MAP-S neat or in combination with a pharmaceutically acceptable carrier are used as a pharmaceutical composition to improve wound healing. The MAP2, MAP4 or MAP8 is covalently bonded to substrate S, such as finely powdered hydroxylapatite, etc. This MAP-S is then contacted with a wound, bone break or injury in need of accelerated repair. Enhanced healing with the use of MAP-S is observed.

### Testing of a MAP-S Structure and its Properties

Example 9 below describes the test procedure and results to establish that MAP-S covalent bonding to the surface has occurred. Example 11 also describes the enhanced cell adhesion, migration, proliferation, etc., that is observed in vitro for the MAP structure as compared to uncoated substrate surface and the single strand of for example P-15. These in vitro results are indicative of the same enhanced cell adhesion, etc. that occurs in vivo with MAP-S composites of this invention. Example 12 describes the results of an experiment which shows that smooth muscle cell growth is not enhanced which is another benefit of the MAP structures of the present invention.

Primary Human Umbilical Vein Endothelial Cells (HUVEC) were selected to evaluate peptide coated ePTFE stent graft materials. HUVEC is the most widely used human endothelial cell type in biomaterial research. HUVEC is more sensitive to different surfaces, but less stable and slower in growth than transformed cells. These Experiments are described below.

Two identical experiments discussed below were conducted to make certain that cell growth data were statistically reliable.

The following Examples are to be read as being illustration and exemplary only. They are not to be construed as being limiting in any way or manner.

### Materials

ePTFE graft films with a thickness of 0.002 inch were obtained from Pall Corporation, Port Washington, N.Y.

Cell Culture—HUVEC cells were purchased from Clonetics, Cumbrous Corp., East Rutherford, N.J., Lot 3F0150. Cells were initiated from P2 frozen stock to P3 and P3 cells were seeded on sample films at a density of 10,000 cells/cm². The cell culture assays in the protocol were followed of “In Vitro Study of P-15 and MAP4 Coated ePTFE Stent Graft Materials.”

### Peptide Coating

Peptide coating procedures were followed in the protocol of “In Vitro Study of P-15 and MAP Coated ePTFE Stent Graft Materials.” Both sides of ePTFE films were plasma treated.

### Methods

### Synthesis of MAP Peptides

MAP peptides having fewer than about 100 amino acids (and often less than about 50 amino acids) are synthesized by the conventional Merrifield solid phase peptide synthesis (SPPS) and modifications thereof. The amino acids are sequentially added to a growing chain (see, for example, B. Merrifield, J. Am. Chem. Soc., 85, 2149-56 (1963), (B. Merrifield, 1995) and J. Nestor, et al. U.S. Pat. No. 4,318,905). The basic principle for solid phase peptide synthesis (SPPS) involves the stepwise addition of amino acids to the growing oligopeptide chain that is anchored to a chemically stable solid particle. Thus the particle can be separated from solvents and reagents during its synthesis by simple filtration. Once the synthesis is complete the chain is cleaved from the support and purification takes place in solution.

Automatic peptide synthesis equipment is available from commercial supplies such as Advanced Chemtech, Louisville, Ky., Applied Biosystems, Foster City, Calif. and Beckman Instruments, Inc., Wladwich, N.J.

For example, a MAP4 peptide, (HN₂-Gly-Thr-Pro-Gy-Pro-Gln-Gly-Ile-Ala-Gly-Gil-Arg-Gly-Val-Val-CONH₂)₄-(Lys)₂-Lys-β-Ala-OH was synthesized by a sequence of standard organic chemical reactions using the Merrifield solid phase peptide synthesis (Merrifield, 1995 & Tam, 1995) as is described above and below.

Table 7 below summarizes the details of the synthesis of MAP4 and is adapted for the preparation of MAP structures.

TABLE 7*

| | MAP4 |
|---|---|
| Instrument | Model 90 (Advanced ChemTech) |
| Resin | Fmoc-β-ala-Wang Resin |
| Deprotection | 25% Peperidine/DMF |
| | 1 × 5 min, 1 × 20 min |
| Coupling | DIC |
| Activation | HOBt |
| Monitoring | Kaiser Ninhydrin test |
| Solvent | DMF/DCM/NMP for coupling |
| | DMF/MeOH/DCM for washing |
| Cleavage | TFA:water:triisopropylsilane |
| | 95:2.5:2.5:3 Hours |

*Researchers need to follow or adapt the equipment manufacturer's instructions to produce these and the other polypeptides for use in the present invention.

### Covalent Bonding of Peptides of Substrates (S)

Peptides are covalently bonded to the surface of substrates (S) through a similar plasma method described by M. H. Dang in U.S. Pat. No. 6,159,531 and also in US patent application 20030113478 (2003). In summary, substrates (films, rods or tubes) are treated with atmospheric or low pressure plasma for a period of time at a given intensity. The plasma treated samples are then chemically activated using a mixture solution, such as sodium hydroxide and chloroacetic acid. Peptides, linear or MAP, are covalently bonded to the chemically activated substrates using the well-known coupling agents, such as ethyldiethylaminopropylcarbodiimide (EDC), with or without the addition of N-hydroxysuccinimide (NHS), glutaraldehyde, dimethylpimelidate, dissucinimidyl suberate (DSS) and succinimidyl 4-(N-maleimidomethyl)cycloexane-1-carboxylate (SMCC). The covalent bonding process usually occurs at ambient temperature and slightly acidic conditions.

### Cell Culture

Well-established culture practices are used (see, for example, R. Ian Freshney, Culture of Animal Cells, Wiley-Liss, 2000) to assess the effectiveness of inventive peptides on different substrates. Peptide bonded substrates are die-cut into disks and then attached to the bottom of cell culture plates using medical grade transfer tapes such as 9877 from 3M Corp., St. Paul, Minn. Disk samples are sterilized by ETO or with 70% sterile ethanol for at least two hrs. Primary human cells, such as HUVEC and HSMC, along with the recommended cell culture media are used. Cells are counted at different time point using the Hemocytometer cell counter system from Fisher Scientific, Hampton, N.H.

### Example 1

### Preparation of a MAP4

(NH₂-GTPGPQGIAGQRGVV)₄-(Lys)₂-Lys-β ala-COOH

(a) The MAP4 peptide, (NH₂-Gly-Thr-Pro-Gly-Pro-Gln-Gly-Gln-Arg-Gly-Val-Val)₄-(Lys)₂-Lys-β-Ala-COOH was assembled on a Fmoc-β-Ala Wang resin. Following deprotection of the Fmoc-β-Ala resin, coupling was accomplished with the specific amino acid sequence of the MAP4 peptide. Coupling of all other Fmoc amino acids for the desired sequence was accomplished using 3 equivalents of 1-Hydroxybenzotriazole (HOBt) and 3 equivalents of 1,3 diisopropylcarbodiimide (DIC). Coupling times of about 120 min were employed. After each coupling the protected peptide Wang resin intermediate was washed as before with 3 volumes each of DMF, methanol and DCM. The completeness after the coupling and cleavage reactions were monitored by the Kaiser Ninydrin test. Following the addition of the last Fmoc-residue and following the deprotection of the Fmoc-group, the peptide Wang resin intermediates was again washed with DMF, methanol and DCM, then air dried.

Peptides were cleaved from the resin using a mixture consisting of trifluoroacetic acid:water: triisopropylsilane. (95:2.5:2.5) and stirred for 60 min at ambient temperature. The respective peptides were isolated by precipitation with diethyl ether and dried at room temperature.

Purification and Analysis of Inventive Peptides—Inventive peptides were purified using preparative reverse-phase liquid chromatography (RP-HPLC) on a C-18 support (Detapak 4.0 cm×30 cm, 15μ, 300 Å with a gradient of 0-50% B over 50 min. Buffer A consisted of TFA (0.1%) in water and ‘Buffer B consisted of acetronitrile (with 0.1% TFA). A flow rate of 150 mL/min was employed. Fractions of 1 min (150 mL) were collected. The purity of these fractions was checked by analytical RP-HPLC and fractions containing >95% pure target peptide were cooled and lyophilized overnight. The resulting dried respective peptides were assayed again by analytical RP-HPLC to yield preparations with peptides of >95% target peptides. Finally the purified peptides were analyzed by mass spectrometry (MS) to verify the purity and targeted molecular formula. In some case, peptides are also analyzed by Amino Acid Analysis to verify the ratios of amino acids in the molecule. Typical analytical results are listed in the following Table 8.

TABLE 8
Typical Analytic Results for Inventive Peptides

| | MAP 4 Peptide: |
|---|---|
| | (NH₂-Gly-Thr-Pro-Gly-Pro-Gln- |
| | Gly-Ile-Ala-Gly-Gln-Arg-Gly- |
| | Val-Val)₄-(Lys)₂-Lys-β-Ala- |
| Peptide | COOH |
| Calculated | C₂₅₇H₄₃₅H₈₇H₇₇ |
| Molecular Formula | |
| Theoretical | 5975.87 |
| Molecular Weight | |
| (g/mole) | |
| Found Molecular | 5975 ± 2 |
| Weight by Mass | |
| Spectroscopy | |
| (g/mole) | |
| Purity by Analytical | >95% |
| RP HPLC | |

(a1) Alternatively, the synthesis of this MAP4 structure is accomplished according to the solid phase procedure of J. P. Tam, U.S. Pat. No. 5,580,563 and/or T. Toth, et al. U.S. Pat. No. 5,882,645 using a Fmoc-β-ala-Wang resin. After the deprotection Fmoc-β-ala from the resin, coupling is accomplished as described in Example 1. After the final amino acid residue is added, the Fmoc deprotection group was removed. The Wang resin is washed stepwise with DMF, methanol and DCN. The cleavage of the protecting group and the coupling reaction was monitored using the Kaiser Ninhydrin test. The purified MAP4 peptide produces satisfactory amino acid analysis. MAP4 is then coupled with ePTFE as is described herein.

(b) Similarly, Example 1(a) is repeated except that the cell-binding peptide in MAP is replaced with a stoichiometrically effective amount of RGD. (SEQ ID NO: 2). Improved cell adhesive and cell proliferation are observed.

(c) Similarly, Example 1(a) is repeated except that the cell-binding peptide in MAP is replaced with a stoichiometrically effective amount of YIGSR. (SEQ ID NO: 5). Improved cell adhesive and cell proliferation are observed.

(d) Similarly, Example 1(a) is repeated except that the cell-binding peptide in MAP is replaced with a stoichiometrically effective amount of REDV. (SEQ ID NO: 3). Improved cell adhesive and cell proliferation are observed.

(e) Similarly, Example 1(a) is repeated except that the cell-binding peptide in MAP is replaced with a stoichiometrically effective amount of SIKVAV (SEQ ID NO: 6). Improved cell adhesive and cell proliferation are observed.

(f) Similarly, Example 1(a) is repeated except that the cell-binding peptide in MAP is replaced with a stoichiometrically effective amount of WQPPRAPI (SEQ ID NO: 4). Improved cell adhesive and cell proliferation are observed.

(g) Similarly, Example 1(a) is repeated except that the substrate ePTFE is replaced with a structurally equivalent amount of hydroxyapatite. Improved cell adhesion and cell proliferation are observed.

(h) Similarly, Example 1(a) is repeated except that the substrate ePTFE is replaced with a structurally equivalent amount of titanium alloy. Improved cell adhesion and cell proliferation are observed.

(i) Similarly, Example 1(a) is repeated except that the substrate ePTFE is replaced with a structurally equivalent amount of polyarylsulfone. Improved cell adhesion and cell proliferation are observed.

(j) Similarly, Example 1(a) is repeated except that the substrate ePTFE is replaced with a structurally equivalent amount of polyetherketone. Improved cell adhesion and cell proliferation are observed.

(k) Similarly, Example 1(a) is repeated except that the substrate ePTFE is replaced with a structurally equivalent amount of polyurethane. Improved cell adhesion and cell proliferation are observed.

(l) Similarly, Example 1(a) is repeated except that the substrate ePTFE is replaced with a structurally equivalent amount of polyurethane. Improved cell adhesion and cell proliferation are observed.

(m) Similarly, Example 1(a) is repeated except that the substrate ePTFE is replaced with a structurally equivalent amount of surface treated stainless steel. Improved cell adhesion and cell proliferation are observed.

(n) Similarly, Example 1(a) is repeated except that the substrate ePTFE is replaced with a structurally equivalent amount of HEMA. Improved cell adhesion and cell proliferation are observed.

(o) Similarly, Example 1(a) is repeated except that the substrate ePTFE is replaced with a structurally equivalent amount of poly(glycolide). Improved cell adhesion and cell proliferation are observed.

### Example 2

### Preparation of a MAP2

(NH₂-GTPGPQGIAGQRGVV)₂-Lys-β ala-COOH

(a) The MAP2 peptide, (NH₂-Gly-Thr-Pro-Gly-Pro-Gln-Gly-Gln-Arg-Gly-Val-Val)₂-Lys-β-Ala-COOH was assembled on a Fmoc-β-Ala Wang resin. Following deprotection of the Fmoc-β-Ala resin, coupling was accomplished as described in Example 1, but with the specific amino acid sequence of the MAP4 peptide.

(b) Similarly, Example 2(a) is repeated except that the cell-binding peptide in MAP is replaced with a stoichiometrically effective amount of RGD. (SEQ ID NO: 2). Improved cell adhesive and cell proliferation are observed.

(c) Similarly, Example 2(a) is repeated except that the cell-binding peptide in MAP is replaced with a stoichiometrically effective amount of YIGSR. (SEQ ID NO: 5). Improved cell adhesive and cell proliferation are observed.

(d) Similarly, Example 2(a) is repeated except that the cell-binding peptide in MAP is replaced with a stoichiometrically effective amount of REDV. (SEQ ID NO: 3). Improved cell adhesive and cell proliferation are observed.

(e) Similarly, Example 2(a) is repeated except that the cell-binding peptide in MAP is replaced with a stoichiometrically effective amount of SIKVAV (SEQ ID NO: 6). Improved cell adhesive and cell proliferation are observed.

(f) Similarly, Example 2(a) is repeated except that the cell-binding peptide in MAP is replaced with a stoichiometrically effective amount of WQPPRAPI (SEQ ID NO: 4). Improved cell adhesive and cell proliferation are observed.

(g) Similarly, Example 2(a) is repeated except that the substrate ePTFE is replaced with a structurally equivalent amount of hydroxyapatite. Improved cell adhesion and cell proliferation are observed.

(h) Similarly, Example 2(a) is repeated except that the substrate ePTFE is replaced with a structurally equivalent amount of titanium alloy. Improved cell adhesion and cell proliferation are observed.

(i) Similarly, Example 2(a) is repeated except that the substrate ePTFE is replaced with a structurally equivalent amount of polyarylsulfone. Improved cell adhesion and cell proliferation are observed.

(j) Similarly, Example 2(a) is repeated except that the substrate ePTFE is replaced with a structurally equivalent amount of polyetherketone. Improved cell adhesion and cell proliferation are observed.

(k) Similarly, Example 2(a) is repeated except that the substrate ePTFE is replaced with a structurally equivalent amount of polyurethane. Improved cell adhesion and cell proliferation are observed.

(l) Similarly, Example 2(a) is repeated except that the substrate ePTFE is replaced with a structurally equivalent amount of polyurethane. Improved cell adhesion and cell proliferation are observed.

(m) Similarly, Example 2(a) is repeated except that the substrate ePTFE is replaced with a structurally equivalent amount of surface treated stainless steel. Improved cell adhesion and cell proliferation are observed.

(n) Similarly, Example 2(a) is repeated except that the substrate ePTFE is replaced with a structurally equivalent amount of HEMA. Improved cell adhesion and cell proliferation are observed.

(o) Similarly, Example 2(a) is repeated except that the substrate ePTFE is replaced with a structurally equivalent amount of poly(glycolide). Improved cell adhesion and cell proliferation are observed.

### Example 3

### Preparation of a MAP4

(CH₃CO-GTPGPQGIAGQRGVV)₄-(Lys)₂-Lys(NH₂)-β ala-
CONH₂

(a) The MAP4 peptide, (CH₃CO-Gly-Thr-Pro-Gly-Pro-Gln-Gly-Gln-Arg-Gly-Val-Val)₄-(Lys)₂-Lys-(NH₂)-β-Ala-CO NH₂ was assembled on a Fmoc-β-Ala Wang resin. A procedure similar to that of Example 1(a) was used. An additional lysine group was added to the Fmoc-β ala resin. The N-terminal group was protected by acetylation. The purified MAP4 produces satisfactory amino acid analysis. MAP 4 is then coupled with ePTFE.

(b) Similarly, Example 3(a) is repeated except that Gly-Thr-Pro-Gly-Pro-Gln-Gly-Gln-Arg-Gly-Val-Val (SEQ ID NO: 1) in MAP is replaced with a stoichiometrically effective amount of RGD (SEQ ID NO: 2). Improved cell adhesive and cell proliferation are observed.

(c) Similarly, Example 3(a) is repeated except that Gly-Thr-Pro-Gly-Pro-Gln-Gly-Gln-Arg-Gly-Val-Val (SEQ ID NO: 1) in MAP is replaced with a stoichiometrically effective amount of YIGSR (SEQ ID NO: 5). Improved cell adhesive and cell proliferation are observed.

(d) Similarly, Example 3(a) is repeated except that Gly-Thr-Pro-Gly-Pro-Gln-Gly-Gln-Arg-Gly-Val-Val (SEQ ID NO: 1) in MAP is replaced with a stoichiometrically effective amount of REDV (SEQ ID NO: 3). Improved cell adhesive and cell proliferation are observed.

(e) Similarly, Example 3(a) is repeated except that Gly-Thr-Pro-Gly-Pro-Gln-Gly-Gln-Arg-Gly-Val-Val (SEQ ID NO: 1) in MAP is replaced with a stoichiometrically effective amount of SIKVAV (SEQ ID NO: 6). Improved cell adhesive and cell proliferation are observed.

(f) Similarly, Example 3(a) is repeated except that Gly-Thr-Pro-Gly-Pro-Gln-Gly-Gln-Arg-Gly-Val-Val (SEQ ID NO: 1) in MAP is replaced with a stoichiometrically effective amount of WQPPRAPI (SEQ ID NO: 4). Improved cell adhesive and cell proliferation are observed.

(g) Similarly, Example 3(a) is repeated except that the substrate ePTFE is replaced with a structurally equivalent amount of hydroxyapatite. Improved cell adhesion and cell proliferation are observed.

(h) Similarly, Example 3(a) is repeated except that the substrate ePTFE is replaced with a structurally equivalent amount of titanium alloy. Improved cell adhesion and cell proliferation are observed.

(i) Similarly, Example 3(a) is repeated except that the substrate ePTFE is replaced with a structurally equivalent amount of polyarylsulfone. Improved cell adhesion and cell proliferation are observed.

(j) Similarly, Example 3(a) is repeated except that the substrate ePTFE is replaced with a structurally equivalent amount of polyetherketone. Improved cell adhesion and cell proliferation are observed.

(k) Similarly, Example 3(a) is repeated except that the substrate ePTFE is replaced with a structurally equivalent amount of polyurethane. Improved cell adhesion and cell proliferation are observed.

(l) Similarly, Example 3(a) is repeated except that the substrate ePTFE is replaced with a structurally equivalent amount of polyurethane. Improved cell adhesion and cell proliferation are observed.

(m) Similarly, Example 3(a) is repeated except that the substrate ePTFE is replaced with a structurally equivalent amount of surface treated PTFE Improved cell adhesion and cell proliferation are observed.

(n) Similarly, Example 3(a) is repeated except that the substrate ePTFE is replaced with a structurally equivalent amount of HEMA. Improved cell adhesion and cell proliferation are observed.

(o) Similarly, Example 3(a) is repeated except that the substrate ePTFE is replaced with a structurally equivalent amount of poly(glycolide). Improved cell adhesion and cell proliferation are observed.

### Example 4

### Preparation of a MAP2

(CH₃CO-GTPGPQGIAGQRGVV)₂-(Lys)-Lys(NH₂)-β-ala-
COOH

(a) The MAP2 peptide, (CH₃CO-Gly-Thr-Pro-Gly-Pro-Gln-Gly-Gln-Arg-Gly-Val-Val)₄-(Lys)-Lys(NH₂)-β-ala-COOH is assembled on a Fmoc-β-Ala Wang resin. A procedure similar to that of Example 3(a) is used. An additional lysine group is added to the Fmoc-β ala resin. The N-terminal group is protected by acetylation. The purified MAP4 produces satisfactory amino acid analysis. MAP 4 is then coupled with ePTFE.

(b) Similarly, Example 4(a) is repeated except that Gly-Thr-Pro-Gly-Pro-Gln-Gly-Gln-Arg-Gly-Val-Val (SEQ ID NO: 1) in MAP is replaced with a stoichiometrically effective amount of RGD (SEQ ID NO: 2). Improved cell adhesive and cell proliferation are observed.

(c) Similarly, Example 4(a) is repeated except that Gly-Thr-Pro-Gly-Pro-Gln-Gly-Gln-Arg-Gly-Val-Val (SEQ ID NO: 1) in MAP is replaced with a stoichiometrically effective amount of YIGSR (SEQ ID NO: 5). Improved cell adhesive and cell proliferation are observed.

(d) Similarly, Example 4(a) is repeated except that Gly-Thr-Pro-Gly-Pro-Gln-Gly-Gln-Arg-Gly-Val-Val (SEQ ID NO: 1) in MAP is replaced with a stoichiometrically effective amount of REDV (SEQ ID NO: 3). Improved cell adhesive and cell proliferation are observed.

(e) Similarly, Example 4(a) is repeated except that Gly-Thr-Pro-Gly-Pro-Gln-Gly-Gln-Arg-Gly-Val-Val (SEQ ID NO: 1) in MAP is replaced with a stoichiometrically effective amount of SIKVAV (SEQ ID NO: 6). Improved cell adhesive and cell proliferation are observed.

(f) Similarly, Example 4(a) is repeated except that Gly-Thr-Pro-Gly-Pro-Gln-Gly-Gln-Arg-Gly-Val-Val (SEQ ID NO: 1) in MAP is replaced with a stoichiometrically effective amount of WQPPRAPI (SEQ ID NO: 4). Improved cell adhesive and cell proliferation are observed.

(g) Similarly, Example 4(a) is repeated except that the substrate ePTFE is replaced with a structurally equivalent amount of hydroxyapatite. Improved cell adhesion and cell proliferation are observed.

(h) Similarly, Example 4(a) is repeated except that the substrate ePTFE is replaced with a structurally equivalent amount of titanium alloy. Improved cell adhesion and cell proliferation are observed.

(i) Similarly, Example 4(a) is repeated except that the substrate ePTFE is replaced with a structurally equivalent amount of polyarylsulfone. Improved cell adhesion and cell proliferation are observed.

(j) Similarly, Example 4(a) is repeated except that the substrate ePTFE is replaced with a structurally equivalent amount of polyetherketone. Improved cell adhesion and cell proliferation are observed.

(k) Similarly, Example 4(a) is repeated except that the substrate ePTFE is replaced with a structurally equivalent amount of polyurethane. Improved cell adhesion and cell proliferation are observed.

(l) Similarly, Example 4(a) is repeated except that the substrate ePTFE is replaced with a structurally equivalent amount of polyurethane. Improved cell adhesion and cell proliferation are observed.

(m) Similarly, Example 4(a) is repeated except that the substrate ePTFE is replaced with a structurally equivalent amount of surface treated PTFE Improved cell adhesion and cell proliferation are observed.

(n) Similarly, Example 4(a) is repeated except that the substrate ePTFE is replaced with a structurally equivalent amount of HEMA. Improved cell adhesion and cell proliferation are observed.

(o) Similarly, Example 4(a) is repeated except that the substrate ePTFE is replaced with a structurally equivalent amount of poly(glycolide). Improved cell adhesion and cell proliferation are observed.

### Example 5

### Preparation of a MAP4

(CH₃CO-GTPGPQGIAGQRGVV)₄-(Lys)₂-Lys(NH₂)-β ala-
COOH

(a) A similar procedure as in Example 4 (a) was used in the preparation of (CH₃CO-Gly-Thr-Pro-Gly-Pro-Gln-Gly-Gln-Arg-Gly-Val-Val)₄-(Lys)₂-Lys(NH₂)-β-Ala-COOH. An additional lysine group was added to the Fmoc-β-ala resin. The purified MAP4 produces satisfactory amino acid analysis. The MAP was then coupled with ePTFE.

(b) Similarly, Example 5(a) is repeated except that the cell-binding peptide in MAP is replaced with a stoichiometrically effective amount of RGD. (SEQ ID NO: 2). Improved cell adhesive and cell proliferation are observed.

(c) Similarly, Example 5(a) is repeated except that the cell-binding peptide in MAP is replaced with a stoichiometrically effective amount of YIGSR. (SEQ ID NO: 5). Improved cell adhesive and cell proliferation are observed.

(d) Similarly, Example 5(a) is repeated except that the cell-binding peptide in MAP is replaced with a stoichiometrically effective amount of REDV. (SEQ ID NO: 3). Improved cell adhesive and cell proliferation are observed.

(e) Similarly, Example 5(a) is repeated except that the cell-binding peptide in MAP is replaced with a stoichiometrically effective amount of SIKVAV (SEQ ID NO: 6). Improved cell adhesive and cell proliferation are observed.

(f) Similarly, Example 5(a) is repeated except that the cell-binding peptide in MAP is replaced with a stoichiometrically effective amount of WQPPRAPI (SEQ ID NO: 4). Improved cell adhesive and cell proliferation are observed.

(g) Similarly, Example 5(a) is repeated except that the substrate ePTFE is replaced with a structurally equivalent amount of hydroxyapatite. Improved cell adhesion and cell proliferation are observed.

(h) Similarly, Example 5(a) is repeated except that the substrate ePTFE is replaced with a structurally equivalent amount of titanium alloy. Improved cell adhesion and cell proliferation are observed.

(i) Similarly, Example 5(a) is repeated except that the substrate ePTFE is replaced with a structurally equivalent amount of polyarylsulfone. Improved cell adhesion and cell proliferation are observed.

(j) Similarly, Example 5(a) is repeated except that the substrate ePTFE is replaced with a structurally equivalent amount of polyetherketone. Improved cell adhesion and cell proliferation are observed.

(k) Similarly, Example 5(a) is repeated except that the substrate ePTFE is replaced with a structurally equivalent amount of polyurethane. Improved cell adhesion and cell proliferation are observed.

(l) Similarly, Example 5(a) is repeated except that the substrate ePTFE is replaced with a structurally equivalent amount of polyurethane. Improved cell adhesion and cell proliferation are observed.

(m) Similarly, Example 5(a) is repeated except that the substrate ePTFE is replaced with a structurally equivalent amount of surface treated stainless steel. Improved cell adhesion and cell proliferation are observed.

(n) Similarly, Example 5(a) is repeated except that the substrate ePTFE is replaced with a structurally equivalent amount of HEMA. Improved cell adhesion and cell proliferation are observed.

(o) Similarly, Example 5(a) is repeated except that the substrate ePTFE is replaced with a structurally equivalent amount of poly(glycolide). Improved cell adhesion and cell proliferation are observed.

### Example 6

### Preparation of a MAP8

(NH₂-GTPGPQGIAGQRGVV)₈-(Lys)₄-(Lys)₂-Lys-Lys-β
ala-COOH

(a) A procedure similar to that found in Example 1 (a) is used to prepare MAP peptide, (NH₂-GTPGPQGIAGQRGVV)₈-(Lys)₄-(Lys)₂-Lys-β-Ala-COOH assembled on a Fmoc-β-Ala Wang resin. After the final amino acid residue is added and the Fmoc deprotection group removed, the peptide Wang resin is washed with DMF, methanol and DCM. Cleavage of the protecting groups and coupling reactions are also monitored using the Kaiser Ninhydrin test. The purified MAP peptide produces satisfactory amino analyses.

A similar procedure as in Example 4 is used in the preparation of (CH₃CO-Gly-Thr-Pro-Gly-Pro-Gln-Gly-Gln-Arg-Gly-Val-Val)₈-(Lys)₄-(Lys)₂-Lys(NH₂)-β-Ala-COOH. An additional lysine group is added to Fmoc-β-Ala Wang resin. The purified MAP8 produces satisfactory amino analysis. The MAP is then coupled with ePTFE.

(b) Similarly, Example 6(a) is repeated except that the cell-binding peptide in MAP is replaced with a stoichiometrically effective amount of RGD. (SEQ ID NO: 2). Improved cell adhesive and cell proliferation are observed.

(c) Similarly, Example 6(a) is repeated except that the cell-binding peptide in MAP is replaced with a stoichiometrically effective amount of YIGSR. (SEQ ID NO: 5). Improved cell adhesive and cell proliferation are observed.

(d) Similarly, Example 6(a) is repeated except that the cell-binding peptide in MAP is replaced with a stoichiometrically effective amount of REDV. (SEQ ID NO: 3). Improved cell adhesive and cell proliferation are observed.

(e) Similarly, Example 6(a) is repeated except that the cell-binding peptide in MAP is replaced with a stoichiometrically effective amount of SIKVAV (SEQ ID NO: 6). Improved cell adhesive and cell proliferation are observed.

(f) Similarly, Example 6(a) is repeated except that the cell-binding peptide in MAP is replaced with a stoichiometrically effective amount of WQPPRAPI (SEQ ID NO: 4). Improved cell adhesive and cell proliferation are observed.

(g) Similarly, Example 6(a) is repeated except that the substrate ePTFE is replaced with a structurally equivalent amount of hydroxyapatite. Improved cell adhesion and cell proliferation are observed.

(h) Similarly, Example 6(a) is repeated except that the substrate ePTFE is replaced with a structurally equivalent amount of titanium alloy. Improved cell adhesion and cell proliferation are observed.

(i) Similarly, Example 6(a) is repeated except that the substrate ePTFE is replaced with a structurally equivalent amount of polyarylsulfone. Improved cell adhesion and cell proliferation are observed.

(j) Similarly, Example 6(a) is repeated except that the substrate ePTFE is replaced with a structurally equivalent amount of polyetherketone. Improved cell adhesion and cell proliferation are observed.

(k) Similarly, Example 6(a) is repeated except that the substrate ePTFE is replaced with a structurally equivalent amount of polyurethane. Improved cell adhesion and cell proliferation are observed.

(l) Similarly, Example 6(a) is repeated except that the substrate ePTFE is replaced with a structurally equivalent amount of polyurethane. Improved cell adhesion and cell proliferation are observed.

(m) Similarly, Example 6(a) is repeated except that the substrate ePTFE is replaced with a structurally equivalent amount of surface treated stainless steel. Improved cell adhesion and cell proliferation are observed.

(n) Similarly, Example 6(a) is repeated except that the substrate ePTFE is replaced with a structurally equivalent amount of HEMA. Improved cell adhesion and cell proliferation are observed.

(o) Similarly, Example 6(a) is repeated except that the substrate ePTFE is replaced with a structurally equivalent amount of poly(glycolide). Improved cell adhesion and cell proliferation are observed.

### Example 7

### Preparation of a MAP8

(CH₃CO-GTPGPQGIAGQRGVV)₈-(Lys)₄-(Lys)₂-Lys(NH₂)-
β-Ala-COOH

(a) A similar procedure as in Example 4 is used in the preparation of (CH₃CO-Gly-Thr-Pro-Gly-Pro-Gln-Gly-Gln-Arg-Gly-Val-Val)₈-(Lys)₄-(Lys)₂-Lys-Lys(NH₂)-β-Ala-COOH. In this case, the MAP has eight arms. The purified MAP8 produces satisfactory amino analysis. MAP is then coupled with ePTFE as described herein.

(b) Similarly, Example 7(a) is repeated except that the cell-binding peptide in MAP is replaced with a stoichiometrically effective amount of RGD. (SEQ ID NO: 2). Improved cell adhesive and cell proliferation are observed.

(c) Similarly, Example 7(a) is repeated except that the cell-binding peptide in MAP is replaced with a stoichiometrically effective amount of YIGSR. (SEQ ID NO: 5). Improved cell adhesive and cell proliferation are observed.

(d) Similarly, Example 7(a) is repeated except that the cell-binding peptide in MAP is replaced with a stochiometrically effective amount of REDV. (SEQ ID NO: 3). Improved cell adhesive and cell proliferation are observed.

(e) Similarly, Example 7(a) is repeated except that the cell-binding peptide in MAP is replaced with a stoichiometrically effective amount of SIKVAV (SEQ ID NO: 6). Improved cell adhesive and cell proliferation are observed.

(f) Similarly, Example 7(a) is repeated except that the cell-binding peptide in MAP is replaced with a stoichiometrically effective amount of WQPPRAPI (SEQ ID NO: 4). Improved cell adhesive and cell proliferation are observed.

(g) Similarly, Example 7(a) is repeated except that the substrate ePTFE is replaced with a structurally equivalent amount of hydroxyapatite. Improved cell adhesion and cell proliferation are observed.

(h) Similarly, Example 7(a) is repeated except that the substrate ePTFE is replaced with a structurally equivalent amount of titanium alloy. Improved cell adhesion and cell proliferation are observed.

(i) Similarly, Example 7(a) is repeated except that the substrate ePTFE is replaced with a structurally equivalent amount of polyarylsulfone. Improved cell adhesion and cell proliferation are observed.

(j) Similarly, Example 7(a) is repeated except that the substrate ePTFE is replaced with a structurally equivalent amount of polyetherketone. Improved cell adhesion and cell proliferation are observed.

(k) Similarly, Example 7(a) is repeated except that the substrate ePTFE is replaced with a structurally equivalent amount of polyurethane. Improved cell adhesion and cell proliferation are observed.

(l) Similarly, Example 7(a) is repeated except that the substrate ePTFE is replaced with a structurally equivalent amount of polyurethane. Improved cell adhesion and cell proliferation are observed.

(m) Similarly, Example 7(a) is repeated except that the substrate ePTFE is replaced with a structurally equivalent amount of surface treated stainless steel. Improved cell adhesion and cell proliferation are observed.

(n) Similarly, Example 7(a) is repeated except that the substrate ePTFE is replaced with a structurally equivalent amount of HEMA. Improved cell adhesion and cell proliferation are observed.

(o) Similarly, Example 7(a) is repeated except that the substrate ePTFE is replaced with a structurally equivalent amount of poly(glycolide). Improved cell adhesion and cell proliferation are observed.

### Example 8

### Preparation of

(CH₃CO-GTPGPQGIAGQRGVV)₈-(Lys)₄-(Lys)₂-Lys-Lys-
(NH₂)-β-Ala-CONH₂

(a) The MAP8 peptide, (CH₃CO-Gly-Thr-Pro-Gly-Pro-Gln-Gly-Gln-Arg-Gly-Val-Val)₈-(Lys)₄-(Lys)₂-Lys-Lys-(NH₂)-β-Ala-COOH is assembled on a Fmoc-β-Ala Wang resin. Following deprotection of the Fmoc-β-Ala resin, coupling is accomplished as described in Example 1, but with the specific amino acid sequence of the MAP peptide. After the final amino acid residue is added and the Fmoc deprotection group removed, the peptide Wang resin is washed with DMF, methanol and DCM. Cleavage of the protecting groups and coupling reactions are also monitored using the Kaiser Ninhydrin test. The purified MAP8 produces satisfactory amino acid analysis. The MAP is then coupled with ePTFE as described herein.

(b) Similarly, Example 8(a) is repeated except that the cell binding peptide in MAP is replaced with a stoichiometrically effective amount of RGD (SEQ ID NO: 2). Improved cell adhesive and cell proliferation are observed when it is coupled with ePTFE.

(c) Similarly, Example 8(a) is repeated except that the cell binding peptide in MAP is replaced with a stoichiometrically effective amount of YIGSR (SEQ ID NO: 5). Improved cell adhesive and cell proliferation are observed when it is coupled with ePTFE.

(d) Similarly, Example 8(a) is repeated except that the cell binding peptide in MAP replaced with a stoichiometrically effective amount of REDV (SEQ ID NO: 3). Improved cell adhesive and cell proliferation are observed when it is coupled with polysulfone.

(e) Similarly, Example 8(a) is repeated except that the cell binding peptide in MAP is replaced with a stoichiometrically effective amount of SIKVAV (SEQ ID NO: 4). Improved cell adhesive and cell proliferation are observed when it is coupled with titanium alloy.

(f) Similarly, Example 8(a) is repeated except that the cell-binding peptide in MAP8 is replaced with a stoichiometrically effective amount of WQPPRAPI (SEQ ID NO: 4). Improved cell adhesive and cell proliferation are observed when it is coupled with stainless steel.

(g) Similarly, Example 8(a) is repeated except that the substrate ePTFE is replaced with a structurally equivalent amount of hydroxyapatite. Improved cell adhesion and cell proliferation are observed.

(h) Similarly, Example 8(a) is repeated except that the substrate ePTFE is replaced with a structurally equivalent amount of titanium alloy. Improved cell adhesion and cell proliferation are observed.

(i) Similarly, Example 8(a) is repeated except that the substrate ePTFE is replaced with a structurally equivalent amount of polyarylsulfone. Improved cell adhesion and cell proliferation are observed.

(j) Similarly, Example 8(a) is repeated except that the substrate ePTFE is replaced with a structurally equivalent amount of polyetherketone. Improved cell adhesion and cell proliferation are observed.

(k) Similarly, Example 8(a) is repeated except that the substrate ePTFE is replaced with a structurally equivalent amount of polyurethane. Improved cell adhesion and cell proliferation are observed.

(l) Similarly, Example 8(a) is repeated except that the substrate ePTFE is replaced with a structurally equivalent amount of polyurethane. Improved cell adhesion and cell proliferation are observed.

(m) Similarly, Example 8(a) is repeated except that the substrate ePTFE is replaced with a structurally equivalent amount of surface treated PTFE Improved cell adhesion and cell proliferation are observed.

(n) Similarly, Example 8(a) is repeated except that the substrate ePTFE is replaced with a structurally equivalent amount of HEMA. Improved cell adhesion and cell proliferation are observed.

(o) Similarly, Example 8(a) is repeated except that the substrate ePTFE is replaced with a structurally equivalent amount of poly(glycolide). Improved cell adhesion and cell proliferation are observed.

### Example 9

### A Map 16

### Preparation of

(CH₃CO-GTPGPQGIAGQRGVV)₁₆-(Lys)₈-(Lys)₄-(Lys)₂-
Lys-Lys(NH₂)-β-Ala-COOH

(a) The MAP16 peptide, (CH₃CO-Gly-Thr-Pro-Gly-Pro-Gln-Gly-Gln-Arg-Gly-Val-Val)₁₆-(Lys)₈-(Lys)₄-(Lys)₂-Lys-Lys(NH₂)-β-Ala-COOH is assembled on a Fmoc-β-Ala Wang resin. Following deprotection of the Fmoc-β-Ala resin, coupling is accomplished as described in Example 1, but with the specific amino acid sequence of the MAP peptide. After the final amino acid residue is added and the Fmoc deprotection group removed, the peptide Wang resin is washed with DMF, methanol and DCM. Cleavage of the protecting groups and coupling reactions are also monitored using the Kaiser Ninhydrin test. The purified MAP4 produces satisfactory amino acid analysis.

(b) Similarly, Example 9(a) is repeated except that the cell-binding peptide in MAP16 is replaced with a stoichiometrically effective amount of RGD (SEQ ID NO: 2). Improved cell adhesive and cell proliferation are observed when it is coupled with ePTFE.

(c) Similarly, Example 9(a) is repeated except that the cell-binding peptide in MAP 16 is replaced with a stoichiometrically effective amount of YIGSR (SEQ ID NO: 5). Improved cell adhesive and cell proliferation are

observed when it is

coupled with ePTFE.

(d) Similarly, Example 9(a) is repeated except that the cell-binding peptide in MAP 16 is replaced with a stoichiometrically effective amount of REDV (SEQ ID NO: 3). Improved cell adhesive and cell proliferation are observed when it is coupled with polysulfone.

(e) Similarly, Example 9(a) is repeated except that the cell-binding peptide in MAP16 is replaced with a stoichiometrically effective amount of SIKVAV (SEQ ID NO: 6). Improved cell adhesive and cell proliferation are observed when it is coupled with titanium alloy.

(f) Similarly, Example 9(a) is repeated except that the cell-binding peptide in MAP 16 is replaced with a stoichiometrically effective amount of WQPPRAPI (SEQ ID NO: 4). Improved cell adhesive and cell proliferation are observed when it is coupled with stainless steel.

(g) Similarly, Example 9(a) is repeated except that the substrate ePTFE is replaced with a structurally equivalent amount of HEMA. Improved cell adhesion and cell proliferation are observed.

(h) Similarly, Example 9(a) is repeated except that the substrate ePTFE is replaced with a structurally equivalent amount of poly(glycolide). Improved cell adhesion and cell proliferation are observed.

### Example 9A

### Preparation of-MAP8-Anti-Inflammatory Agent

(Naproxen)₈-(Lys)₄-(Lys)₂-Lys-Lys-(NH₂)-β-Ala-
CONH₂

(a) The MAP8 peptide, (Naproxen)₈-(Lys)₄-(Lys)₂-Lys-Lys-(NH₂)-β-Ala-COOH is assembled on a Fmoc-β-Ala Wang resin. Following deprotection of the Fmoc-β-Ala resin, coupling is accomplished as described in Example 1(a), but with naproxen-amino acid sequence of the MAP peptide. After the final amino acid residue is added and the Fmoc deprotection group removed, the peptide Wang resin is washed with DMF, methanol and DCM. Cleavage of the protecting groups and coupling reactions are also monitored using the Kaiser Ninhydrin test. The purified MAP8 produces satisfactory amino acid analysis. The MAP is then coupled with ePTFE as described herein. This MAP-S when prepared as described in Example 1(g) to 1(o) exhibits enhanced anti-inflammatory properties.

### Example 9B

### Preparation of-MAP8-Growth Factor

(Growth factor-VEGF)₈-(Lys)₄-(Lys)₂-Lys-Lys-(NH₂)-
β-Ala-CONH₂

(a) The MAP8 peptide, (Growth Factor-VEGF)₈-(Lys)₄-(Lys)₂-Lys-Lys-(NH₂)-β-Ala-COOH is assembled on a Fmoc-β-Ala Wang resin. Following deprotection of the Fmoc-β-Ala resin, coupling is accomplished as described in Example 1(a), but with the specific growth factor—VEGF amino acid sequence of the MAP peptide. After the final amino acid residue is added and the Fmoc deprotection group removed, the peptide Wang resin is washed with DMF, methanol and DCM. Cleavage of the protecting groups and coupling reactions are also monitored using the Kaiser Ninhydrin test. The purified MAP8 produces satisfactory amino acid analysis. The MAP is then coupled with ePTFE as described herein. The MAP-S when prepared as described in Example 1(g) to 1(o) exhibits enhanced growth factor properties.

### Example 9C

### Preparation of-MAP8-Adhesion Barrier

(Adhesion barrier-oligomer)₈-(Lys)₄(Lys)₂-Lys-Lys-(NH₂)-β-Ala-CONH₂

(a) The MAP8 peptide, (Adhesion barrier-oligomer)₈-(Lys)₄-(Lys)₂-Lys-Lys-(NH₂)-β-Ala-COOH is assembled on a Fmoc-β-Ala Wang resin. Following deprotection of the Fmoc-β-Ala resin, coupling is accomplished as described in Example 1(a), but with the specific adhesion barrier oligomer such as SEPRAFILM as ligand of the MAP peptide. After the final amino acid residue is added and the Fmoc deprotection group removed, the peptide Wang resin was washed with DMF, methanol and DCM. Cleavage of the protecting groups and coupling reactions were also monitored using the Kaiser Ninhydrin test. The purified MAP8 produces satisfactory amino acid analysis. The MAP is then coupled with ePTFE as described herein. This MAP-S when prepared as described in Example 1(g) and 1(o) exhibits enhanced adhesion barrier properties.

### Example 10

### MAP40F RGD & Naproxen

RGD-COOH as described herein is combined with an equimolar amount of naproxen (d-2-(6-methoxyl-2-naphlyl) propionic acid) and is combined under Merrifield solid state synthesis conditions with one equivalent of (NH₂Lys)₄-(Lys)₂-Lys-β-ala solid polymer. After the normal reaction times described herein, the MAP is cleaved from the substrate in the conventional way and purified. A MAP4 having ligands about in the 50/50 ratio of about RGD/naproxen is obtained.

### Example 11

### Huvec Cells

(a) In this example ePTFE films covalently coated with MAP4 peptide (NH₂-GTPGPQGIAGQRGVV)₄-(Lys)₂-Lys-β-Ala-COOH and linear peptide NH₂-GTPGPQGIAGQRGVV-CONH₂ (P-15) were evaluated for cell adhesion and proliferation using primary Human Umbilical Vein Endothelial Cells (HUVEC). Two identical experiments were conducted to make certain that the cell growth data were statistically reliable (Exp. A and Exp. B). ePTFE films with a thickness of 0.002″ were obtained form Pall Corporation, Port Washington N.Y. Human Umbilical Vein Endothelial Cells (HUVEC) were purchased from Clonetics Corporation, East Rutherford N.J. Cells were initiated from P2 frozen stock to P3 and P3 cells were seeded on sample films at a density of 10,000 cells/cm² in the EBM medium. Standard cell initiation, seeding, incubation, trypsinization and counting procedures were used in the example (See for example R. Ian Freshney, Culture of Animal Cells, Wiley-Liss, 2000). 12-well plates were used in the cell culture and cells were counted after 24 hrs, 4 days, 7 days, 10 days 14 days and 17 days. After the peptide coating, samples were vigorously wasted with dionized water and ethanol to remove free (unbonded) peptide molecules on the surface.

Peptide coating density on coated ePTFE was evaluated using Amino Acid Analysis (AAA Service Lab, Boring Oregon). The results are listed in Table 8.

TABLE 8
Peptide Coating Density

| | MAP4 | P-15 |
|---|---|---|
| Experiment No. | (nMoles/cm²) | (nMoles/cm²) |
| 11A | 1.2 | 1.7 |
| 11B | 1.3 | 1.3 |

Based on the theoretical calculation, if the surface area of the linear peptide molecule NH₂-GTPGPQGIAGQRGVV-CONH₂ (SEQ ID NO: 1) about 100 Å (very conservative) then a single layer coverage of the linear peptide molecules would require about 0.3 nMoles/cm². The calculation considered the fact that the actual surface area of ePTFE is larger than the measured area because of the porous surface. Since the MAP peptide, (NH₂-Gly-The-Pro-Gly-Pro-Gln-Gly-Ile-Ala-Gly-Gln-Arg-Gly-Val-Val)₄-(Lys)₂-Lys-β-Ala-COOH, is about five times bigger than P-15, even less amount of the peptide is needed to form a single layer on ePTFE.

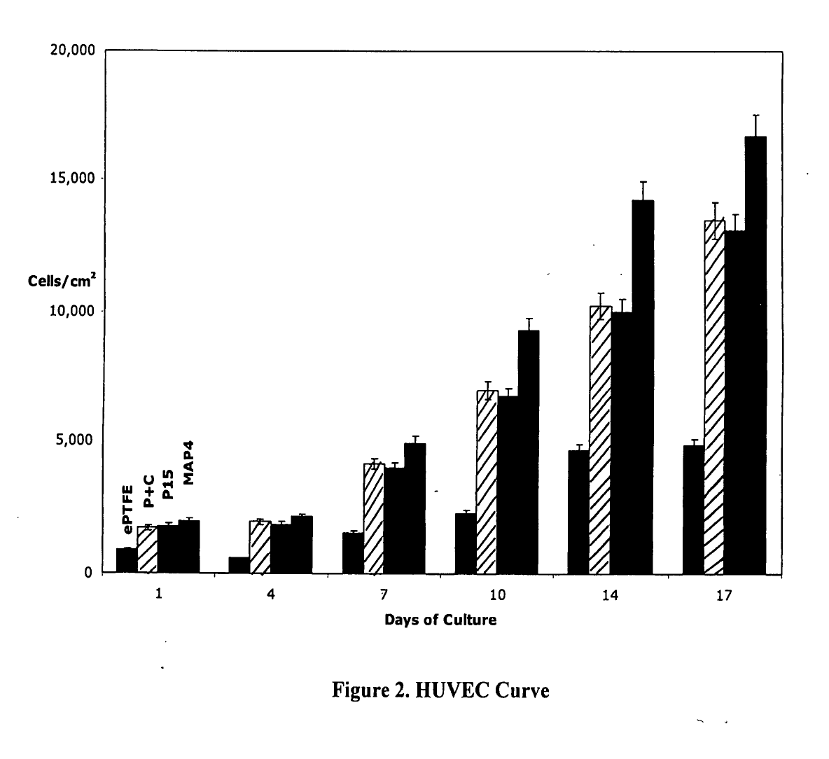

Uncoated ePTFE (ePTFE) and chemical activated (P-1-C) ePTFE were used as controls. For chemical activated samples, ePTFE films were only plasma treated and then chemically activated according to Methods. These samples did not have peptide coatings. Cell growth data were collected as triplicates from three individual wells. Cells were counted after 24 hours, 4 days, 7 days, 10 days, 14 days, and 17 days. Tables 10 and 11 are the cell count results. FIGS. 1 and 2 are the cell growth curves. Cell counts after 24 hrs were used to calculate the average cell adhesion (See Table 9).

TABLE 9
Average Cell Adhesion after 24 Hours

| Sample | Cell Adhesion (%) |
|---|---|
| ePTFE | 8.3 |
| P + C | 16.6 |
| P-15 | 16.9 |
| MAP4 | 21.2 |

The average cell adhesion was 21.2% for MAP4 samples, about 17% for chemical activated and P-15 coated samples, and only 8.3% for ePTFE controls. The MAP peptide coated ePTFE had the largest increase in cell adhesion and cell proliferation over ePTFE control. The cell adhesion of the MAP peptide coated ePTFE after 24 hr was more than 250 percent better than that on ePTFE control. There were 350 percent more living cells on the MAP peptide coated ePTFE than that on ePTFE control after 17 days' incubation. The MAP peptide coated ePTFE was also more cell-promoting than the linear peptide coated ePTFE, about 22 percent more cells on the MAP peptide coated ePTFE. (Tables 10 and 11). Similar cell growth data were found for both the linear peptide coated and chemical-activated ePTFE samples. All cell count data were evaluated by the two-way statistical analysis of variance (Table 12).

The improved cell adhesion data by the MAP peptide coating on ePTFE provided the evidence that MAP peptides are more effective in cell-binding and cell-proliferation than liner peptides with similar cell adhesion domains. In this invention, it is demonstrated that the MAP peptide coating on ePTFE not only improved cell adhesion but also significantly enhanced cell proliferation over the linear peptide alone. The superior cell-promoting properties exhibited by the MAP peptide coating on ePTFE are due to its multiple cell adhesion domains within the MAP peptide molecule and its large size to form suitable orientation and conformation for approaching cells. Both cell adhesion and cell proliferation were significantly improved on the MAP peptide coated ePTFE compared to ePTFE control and the linear peptide coated ePTFE. It appears that the linear peptide coating might have only made the surface of ePTFE more hydrophilic because the cell behaviors on the linear peptide coated samples were similar to that on chemically activated samples. The purpose of the chemical activation step is to introduce carboxylic groups onto the surface of ePTFE.

TABLE 10
Cell Density of Exp 11A*

| | Culture Time (Days) | | | | | |
|---|---|---|---|---|---|---|
| | 1 | 4 | 7 | 10 | 14 | 17 |
| ePTFE | 748 ± 111 | 482 ± 42 | 1,882 ± 191 | 2,721 ± 133 | 5,384 ± 174 | 4,632 ± 383 |
| P + C | 1,616 ± 111 | 2,219 ± 84 | 4,656 ± 151 | 8,009 ± 585 | 11,917 ± 364 | 15,390 ± 301 |
| P-15 | 1,664 ± 145 | 2,243 ± 125 | 4,077 ± 292 | 7,575 ± 301 | 10,469 ± 743 | 14,956 ± 221 |
| MAP4 | 2,268 ± 111 | 2,822 ± 191 | 8,226 ± 471 | 10,421 ± 289 | 16,404 ± 684 | 17,272 ± 301 |

*The data represents the mean cell density (cell/cm²) of three separate well counts and ± standard deviation.

TABLE 11
Cell Density of Exp 11B*

| | Culture Time (Days) | | | | | |
|---|---|---|---|---|---|---|
| | 1 | 4 | 7 | 10 | 14 | 17 |
| ePTFE | 917 ± 111 | 579 ± 72 | 1,544 ± 151 | 2,287 ± 100 | 4,632 ± 133 | 4,825 ± 301 |
| P + C | 1,713 ± 42 | 1,954 ± 72 | 4,125 ± 72 | 6,947 ± 145 | 10,228 ± 442 | 13,461 ± 904 |
| P-15 | 1,785 ± 151 | 1,857 ± 111 | 3,980 ± 145 | 6,706 ± 221 | 9,987 ± 289 | 13,075 ± 442 |
| MAP4 | 1,978 ± 42 | 2,147 ± 42 | 4,921 ± 332 | 9,263 ± 434 | 14,232 ± 585 | 16,645 ± 663 |

*The data represents the mean cell density (cell/cm²) of three separate well counts and ± standard deviation.

TABLE 12
Two-Way Statistical Analysis of Variance of Cell Count Data

| | | p-value | p-value | Significant |
|---|---|---|---|---|
| Experiment | Comparison | (set) | (actual) | Difference |
| 11A | MAP4 and ePTFE | 0.05 | P < 0.001 | Yes |
| | MAP4 and P-15 | 0.05 | P < 0.001 | Yes |
| | P-15 and P + C | 0.05 | P < 0.001 | Yes |
| 11B | MAP4 and ePTFE | 0.05 | P < 0.001 | Yes |
| | MAP4 and P-15 | 0.05 | P < 0.001 | Yes |
| | P-15 and P + C | 0.05 | P < 0.146 | No |

(b) Similarly, Example 11(a) is repeated except that the cell-binding sequence in the MAP peptide is replaced with a stoichiometrically effective amount of RGD (SEQ ID NO: 2). Improved cell adhesive and cell proliferation are observed.

(c) Similarly, Example 11(a) is repeated except that the cell-binding sequence in the MAP peptide is replaced with a stoichiometrically effective amount of YIGSR (SEQ ID NO: 5). Improved cell adhesive and cell proliferation are observed.

(d) Similarly, Example 11(a) is repeated except that the cell-binding sequence in the MAP peptide is replaced with a stoichiometrically effective amount of REDV (SEQ ID NO: 3). Improved cell adhesion and cell proliferation are observed.

(e) Similarly, Example 11(a) is repeated except that the cell-binding sequence in the MAP peptide is replaced with a stoichiometrically effective amount of SIKVAV. Improved cell adhesive and cell proliferation are observed. replaced with a stoichiometrically effective amount of SIKVAV (SEQ ID NO: 6). Improved cell adhesive and cell proliferation are observed.

(f) Similarly, Example 11(a) is repeated except that the cell-binding sequence in the MAP peptide is replaced with a stoichiometrically effective amount of WQPPRAPI (SEQ ID NO: 6). Improved cell adhesion and cell proliferation are observed.

(g) Similarly, Example 11(a) is repeated except that the substrate ePTFE is replaced with a structurally equivalent amount of hydroxyapatite. Improved cell adhesion and cell proliferation are observed.

(h) Similarly, Example 11(a) is repeated except that the substrate ePTFE is replaced with a structurally equivalent amount of titanium alloy. Improved cell adhesion and cell proliferation are observed.

(i) Similarly, Example 11(a) is repeated except that the substrate ePTFE is replaced with a structurally equivalent amount of polyarylsulfone. Improved cell adhesion and cell proliferation are observed.

(j) Similarly, Example 11(a) is repeated except that the substrate ePTFE is replaced with a structurally equivalent amount of polyetherketone. Improved cell adhesion and cell proliferation are observed.

(k) Similarly, Example 11(a) is repeated except that the substrate ePTFE is replaced with a structurally equivalent amount of polyurethane. Improved cell adhesion and cell proliferation are observed.

(l) Similarly, Example 11(a) is repeated except that the substrate ePTFE is replaced with a structurally equivalent amount of polyurethane. Improved cell adhesion and cell proliferation are observed.

(m) Similarly, Example 11(a) is repeated except that the substrate ePTFE is replaced with a structurally equivalent amount of surface treated stainless steel. Improved cell adhesion and cell proliferation are observed.

(n) Similarly, Example 11(a) is repeated except that the substrate ePTFE is replaced with a structurally equivalent amount of HEMA. Improved cell adhesion and cell proliferation are observed.

(o) Similarly, Example 11(a) is repeated except that the substrate ePTFE is replaced with a structurally equivalent amount of poly(glycolide). Improved cell adhesion and cell proliferation are observed.

### Example 12

### Smooth Muscle Cells Evaluation

In this example, Primary Human Smooth Muscle Cells (HSMC) were used to assess the effectiveness of peptide coated ePTFE film. All experimental procedures were similar to Example 1.

After incubating for 24 hours, about 87%, 72%, 68% and 33% of cells survived for MAP4 peptide coated, linear peptide coated, P+C control and ePTFE control, respectively. However, smooth muscle cells did not grow significantly after 4 days' incubation for all samples except ePTFE control. Number of cells on ePTFE control was more than doubled. Cells on other samples increased only about 30%. After 4 days' incubation, smooth muscle cells on MAP4 were only 50 percent more than that on ePTFE control (Table 13 and FIG. 14). However, as discussed in Example 1, 400 percent more endothelial cells on the MAP peptide coated ePTFE than on ePTFE control during the same incubation period. This observation is very significant because the data indicate that this specific MAP peptide is more effective promoting HUVEC than HSMC. In other words, MAP peptides can be optimized for not only have the ability to promote cell growth, but also have the selectivity to attract specific cells. This is the exact required combination of surface properties that artificial implants need to have in order to be effectively integrated with the surrounding tissues, quickly forming a stable endothelial cell lining and slowing down the growth of smooth muscle cells. All experiment data were statistically significant (Table 14).

TABLE 13
Cell Density Experimental Results .

| Culture | | | | |
|---|---|---|---|---|
| Time | | | | |
| (Days) | 1 | 2 | 3 | 4 |
| ePTFE | 7,888 ± 205 | 11,316 ± 558 | 15,263 ± 744 | 17,105 ± 1,116 |
| P + C | 16,355 ± 614 | 23,158 ± 1,116 | 24,079 ± 1675 | 21,053 ± 1,116 |
| P-15 | 17,224 ± 409 | 21,974 ± 558 | 25,789 ± 1,116 | 22,105 ± 1,116 |
| MAP4 | 20,842 ± 409 | 24,079 ± 930 | 26,711 ± 558 | 27,237 ± 558 |

TABLE 14
Two-Way Statistical Analysis of Variance of Cell Count Data

| | | | Significant |
|---|---|---|---|
| Comparison | p-value (set) | p-value (actual) | Difference |
| MAP4 and ePTFE | 0.05 | P < 0.001 | Yes |
| MAP4 and P-15 | 0.05 | P < 0.001 | Yes |
| P-15 and P + C | 0.05 | P < 0.273 | No |

While only a few embodiments of the invention have been shown and described herein, it will become apparent to those skilled in the art that various modifications and changes can be made in the MAP structure and features of the compositions of matters, the pharmaceutical compositions, implants, methods of manufacture, or methods of therapy for in vivo adhesion, migration and proliferation of bioactive molecules and to provide anti-inflammatory and anti-thrombogenic properties without departing from the spirit and scope of the present invention. All such modifications and changes coming with the scope of the appended claims are intended to be carried out thereby.

1.-29. (canceled)

30. An implant comprising a matrix formed of a multiple arm peptide-substrate (MAP-S) formed of a biomaterial coated substrate S and a multiple MAP peptide carried by covalent bonding to the substrate, wherein S is selected from the group consisting of metal, alloy, ceramic, natural polymer, synthetic polymer, bioabsorbable polymer, liquid polymer and combinations and blends thereof, and the organic structure MAP is selected from:
(R)ₙ₊₁—(Z)ₙ—X—
wherein n is selected from 1, 3, 7 or 15, producing the following structures: 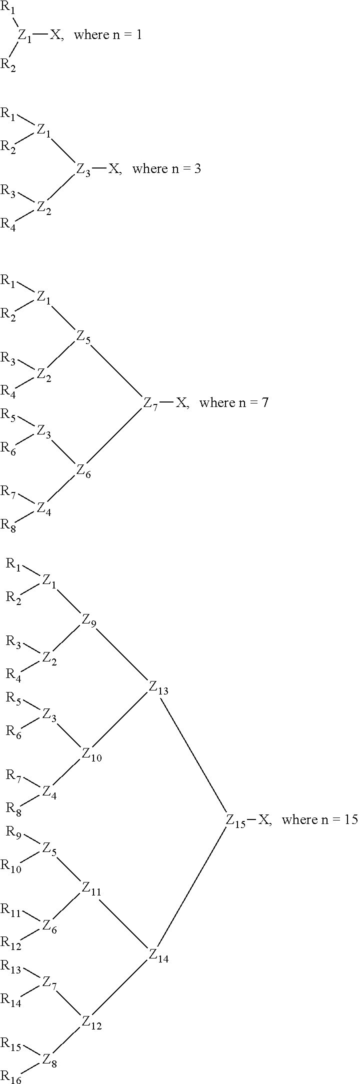
each R contains any type and number of cell-binding ligands, anti-inflammatory structures, anti-thrombogenic structures, growth factor structures, adhesive or adhesion barrier structures, and their combinations, with the proviso that, the MAP has active functional groups to covalently link the MAP structure to the surface of the substrate (S), located on group X, Z or R;
X is an active or protected linking group selected from the group consisting of amine, linked amino acids of 1 to 5 in length, (X1 to X5) which when present are the same or different, carboxylic acid, anhydride, hydroxyl, carbonyl succinimide (NHS) and siloxane;
each Z is independently selected from lysine or ornithine;
each R when present in the MAP structure comprises a total of up to about 100 amino acids, and wherein each R₁ to R₁₆ comprises GTPGPQGIAGQRGVV (SEQ ID NO:1);
wherein the MAP peptide has terminal ligands which have enhanced properties for cell adhesion, migration, cell differentiation, cell proliferation, anti-inflammation, anti-thrombogenesis, cell growth, adhesion barrier and combinations thereof.

31. The implant of claim 30, wherein when implant is exposed to vascular tissue cells, the multiple arm peptide-substrate adheres substantially more cells than the substrate alone.

32. The implant of claim 31, wherein the cells are smooth muscle cells and the multiple arm peptide-substrate adheres about 250% more cells than the substrate alone.

33. The implant of claim 31, wherein the cells are endothelial cells and the multiple arm peptide-substrate adheres about 400% more cells than the substrate alone.

34. The implant of claim 31, wherein the substrate comprises e-PTFE.

35. The implant of claim 30, further comprising living cells on the MAP-S coated surface of the implant.

36. The implant of claim 35, wherein the cells are vascular tissue cells.

37. The implant of claim 35, wherein the cells are endothelial cells.

38. The implant of claim 35, wherein there are substantainly more cells on portions of the substrate containing the MAP peptide.

39. The implant of claim 35, wherein the implant is an in vivo implant.

40. The implant of claim 39, wherein the implant comprises a blood contacting device.

41. The implant of claim 40, wherein the implant comprises a stent.

42. The implant of claim 40, wherein the implant comprises a graft.

43. The implant of claim 39, in which the cells are adhered to the MAP-S coated surface of the implant.

44. The implant of claim 47 which is an in vivo implant.

45. The implant of claim 48, wherein the cells are endothelial cells

46. The implant of claim 48, wherein the cells are smooth muscle cells.

47. A composition of matter for the active structure MAP-S wherein MAP is an organic molecule which is covalently bound to a substrate S, wherein S is selected from the group consisting of metal, alloy, ceramic, natural polymer, synthetic polymer, bioabsorbable polymer, liquid polymer and combinations and blends thereof, and the organic structure MAP is selected from:
(R)ₙ₊₁—(Z)ₙ—X—
wherein n is selected from 1, 3, 7 or 15, producing the following structures: 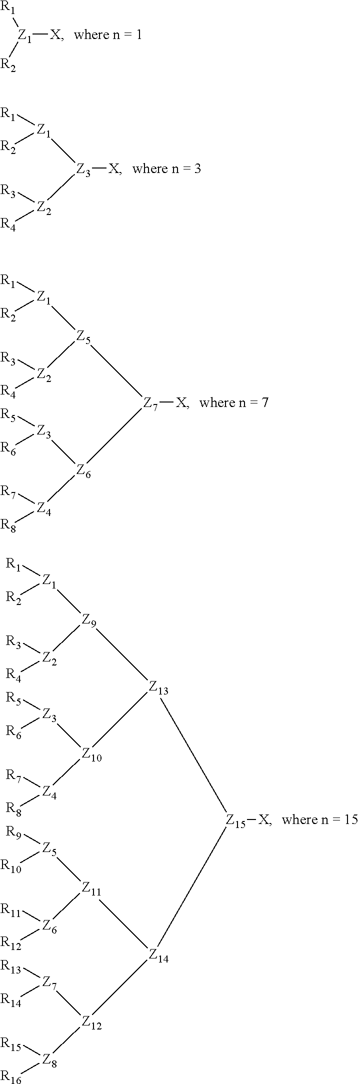
each R contains any type and number of cell-binding ligands, anti-inflammatory structures, anti-thrombogenic structures, growth factor structures, adhesive or adhesion barrier structures, and their combinations, with the proviso that, the MAP has active functional groups to covalently link the MAP structure to the surface of the substrate (S), located on group X, Z or R;
X is an active or protected linking group selected from the group consisting of amine, linked amino acids of 1 to 5 in length, (X1 to X5) which when present are the same or different, carboxylic acid, anhydride, hydroxyl, carbonyl succinimide (NHS) and siloxane;
each Z is independently selected from lysine or ornithine;
each R when present in the MAP structure comprises a total of up to about 100 amino acids, and wherein each R₁ to R₁₆ comprises GTPGPQGIAGQRGVV (SEQ ID NO:1).

48. The composition of matter of claim 47 wherein S is selected from the group consisting of hydroxylapatite, stainless steel, cobalt-chromium-molybdenum alloy, titanium, titanium alloy, polypropylene, polyethylene, polystyrene, polyether, polyamide/polyethylene copolymer, polychloroprene, polyester, polyvinyl chloride, polyolefin, polyphenolic, polyhydroxyacid, ABS epoxy, polytetrafluoroethylene, expanded polytetrafluoroethylene, polytetrafluoroethylene/polyethylene copolymer, fluorinated ethylene propylene, polyvinylidene, hexafluoropropylene, polyurethane, polysiloxane, polyisoprene, silicone, styrene butadiene, natural rubber, latex rubber, polyethyleneterephthalate, polycarbonate, polyamide, polyaramid, polyaryl ether ketone, polyacetal, polyphenylene oxide, polysulfone, polyethersulfone, regenerated cellulose, polyamino acids, polyarylsulfone, polyphenylene sulphide, polybutyl-terephthalate (PBT), poly(glycolide), HEMA and combinations thereof.

49. The composition of matter of claim 47 wherein Z₁ to Z₁₅ is lysine.

50. The composition of matter of claim 47 wherein MAP is MAP4 and R_{I} to R₄ are each independently selected from linear peptides having about 50 amino acids or less.

51. The composition of matter of claim 47 wherein MAP is MAP8 and R₁ to R₈ are each independently selected from linear peptides having about 50 amino acids or less.

52. The composition of matter of claim 47 wherein MAP is MAP16 and R₁ to R₁₆ are each independently selected from linear peptides having about 50 amino acids or less.

53. The composition of matter of claim 47 wherein
S is selected from the group consisting of polytetrafluoroethylene (PTFE) and hydroxylapatite;
X is X₁-X₂, and X₁ and X₂ are selected from the group consisting of carboxyl and amino acid;
Z₁ to Z₁₅ are lysine; and
R₁ to R₁₆ are GTPGPQGIAGQRGVV (SEQ ID NO:1).

54. The composition of matter of claim 47 wherein:
MAP is MAP2 of the structure: 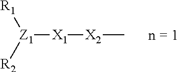
Z₁ is lysine and R₁ and R₂ are each GTPGPQGIAGQRGVV (SEQ ID NO:1);
MAP is MAP4 of the structure: 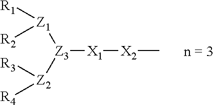
Z₁, Z₂ and Z₃ are lysine and R₁, R₂, R₃ and R₄ are each GTPGPQGIAGQRGVV (SEQ ID NO:1); or
MAP is MAP8 of the structure: 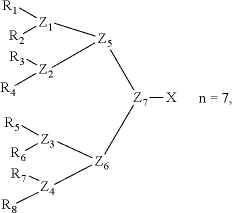
Z₁ to Z₇ are lysine and R₁ to R₈ are each GTPGPQGIAGQRGVV (SEQ ID NO:1); and X is —X₁— or —X₁—X₂— wherein X₁ and X₂ are selected from lysine, ornithine or alanine.

55. A pharmaceutical composition which comprises:
a pharmaceutically acceptable amount of MAP of claim 47 in combination with a pharmaceutically acceptable carrier.

56. A pharmaceutical composition which comprises a pharmaceutically acceptable amount of MAP2, MAP4 or MAP8 of claim 47 with a pharmaceutically acceptable carrier.

57. An implant comprising: a matrix formed of a multiple arm peptide-substrate (MAP-S) formed of a biomaterials coated substrate S and a multiple MAP peptide of claim 47 combined by covalent binding to the substrate, wherein the MAP peptide has terminal ligands which have enhanced properties for cell adhesion, migration, cell differentiation, cell proliferation, anti-inflammation, anti-thrombogenesis, cell growth, adhesion barrier and combinations thereof.

58. The implant of claim 57 wherein the peptide MAP has a peptide sequence selected from the group consisting of MAP ID NO:13, MAP ID NO:14, MAP ID NO:15, MAP ID NO:22, MAP ID NO:23, MAP ID NO:24, MAP ID NO:31, MAP ID NO:32, MAP ID NO:33, MAP ID NO:40, MAP ID NO:41 and MAP ID NO:42.

59. The implant of claim 58 wherein the substrate is selected from polymer materials selected from the group consisting of hydrocarbons, fluorocarbons, elastomers, engineering thermoplastics, and metallic materials.

60. The implant of claim 59 wherein the hydrocarbon polymer material is selected from the group consisting of polypropylene, polyethylene, polystyrene, polyether, polyamide/polyethylene copolymer, polychloroprene, polyester, polyvinyl chloride, polyolefin, polyphenolic, polyhydroxyacid, ABS epoxy, and corresponding copolymers and blends; the fluorocarbon polymer material is selected from the group consisting of polytetrafluoroethylene, expanded polytetrafluoroethylene, polytetrafluoroethylene/polyethylene copolymer, fluorinated ethylene propylene, polyvinylidene fluoride, hexafluoropropylene, and corresponding copolymers and blends; the elastomer polymer material is selected from the group consisting of polyurethane, polysiloxane, polyisoprene, silicone, styrene butadiene, natural rubber, latex rubber, and corresponding copolymers and blends; the engineering thermoplastic polymer material is selected from the group consisting of polyethyleneterephthalate, polycarbonate, polyamide, polyaramid, polyaryl ether ketone, polyacetal, polyphenylene oxide, polysulfone, polyethersulfone, regenerated cellulose, polyamino acids, polyarylsulfone, polyphenylene sulphide, polybutyl-terephthalate (PBT)poly(glycolide), HEMA and corresponding copolymers and blends; and the metallic material is selected form the group consisting of stainless steel, cobalt-chromium-molybdenum alloy, pure titanium, and titanium alloys.

61. The implant of claim 59 wherein:
the peptide MAP is selected from a MAP4 or MAPS.

62. The implant of claim 61 wherein:
R₁ to R₈ when present are GTPGPQGIAGQRGVV (SEQ ID NO:1), Z₁ to Z₇ are lysine, and X₁ and X₂ are selected from β-ala-COON, β-ala-CONH₂, lys, or lys(NH₂).

63. The implant of claim 62 wherein S is selected from the group consisting of e-PTFE, PTFE, polysulfone, polyurethane, silicone, titanium and titanium alloy.

64. The composition of matter of claim 47 wherein each R when present in the MAP structure comprises a total of up to about 50 amino acids, and wherein each R₁ to R₁₆ comprises GTPGPQGIAGQRGVV (SEQ ID NO:1).

65. The composition of matter of claim 47 wherein each R when present in the MAP structure is GTPGPQGIAGQRGVV (SEQ ID NO:1).


SEQUENCE LISTING (13 sequences)

SEQ ID NO: 1 - protein, 15 aa, Artificial Sequence
  Description of Artificial Sequence Synthetic peptide
GTPGPQGIAGQRGVV

SEQ ID NO: 2 - protein, 3 aa, Artificial Sequence
  Description of Artificial Sequence Synthetic peptide
RGD

SEQ ID NO: 3 - protein, 4 aa, Artificial Sequence
  Description of Artificial Sequence Synthetic peptide
REDV

SEQ ID NO: 4 - protein, 8 aa, Artificial Sequence
  Description of Artificial Sequence Synthetic peptide
WQPPRARI

SEQ ID NO: 5 - protein, 5 aa, Artificial Sequence
  Description of Artificial Sequence Synthetic peptide
YIGSR

SEQ ID NO: 6 - protein, 6 aa, Artificial Sequence
  Description of Artificial Sequence Synthetic peptide
SIKVAV

SEQ ID NO: 7 - protein, 20 aa, Artificial Sequence
  Description of Artificial Sequence Synthetic peptide
RYVVLPRPVCFEKGMNYTVR

SEQ ID NO: 8 - protein, 13 aa, Artificial Sequence
  Description of Artificial Sequence Synthetic peptide
GEFYFDLRLKGDK

SEQ ID NO: 9 - protein, 4 aa, Artificial Sequence
  Description of Artificial Sequence Synthetic peptide
GIAG

SEQ ID NO: 10 - protein, 6 aa, Artificial Sequence
  Description of Artificial Sequence Synthetic peptide
QGIAGQ

SEQ ID NO: 11 - protein, 5 aa, Artificial Sequence
  Description of Artificial Sequence Synthetic peptide
KNEED

SEQ ID NO: 12 - protein, 5 aa, Artificial Sequence
  Description of Artificial Sequence Synthetic peptide
PDSGR

SEQ ID NO: 13 - protein, 17 aa, Artificial Sequence
  Description of Artificial Sequence Synthetic peptide
GTPGPQGIAGQRGVVKA